(19) United States
(12) Patent Application Publication
(10) Pub. No.: US 2024/0331158 A1
(43) Pub. Date: Oct. 3, 2024

(54) CELL PAINTING AND ANALYSIS IN 3D CELLULAR MODELS

(71) Applicant: Molecular Devices, LLC, San Jose, CA (US)

(72) Inventors: Angeline Lim, Scotts Valley, CA (US); Oksana Sirenko, San Carlos, CA (US)

(21) Appl. No.: 18/621,726

(22) Filed: Mar. 29, 2024

Related U.S. Application Data
(60) Provisional application No. 63/455,610, filed on Mar. 30, 2023.

(51) Int. Cl. G06T 7/00; G01N 33/50

(52) U.S. Cl. CPC G06T 7/0016; G01N 33/5082; G01N 2800/52; G06T 2207/10064; G06T 2207/30024

(57) ABSTRACT

A Cell Painting method is provided for analysis of 3D tumoroids and evaluation of phenotypic effects. Methods are provided for increased throughput, imaging and automation in 3D assays that are suitable for compound screening using patient-derived samples.

### CROSS-REFERENCES TO RELATED APPLICATIONS

This application claims the benefit of U.S. Provisional Application No. 63/455,610, filed Mar. 30, 2023, which is incorporated herein by reference in its entirety.

### BACKGROUND

There is a critical need to develop methods for efficient testing of drug efficacy in patient-derived tumor samples to discover new therapeutics. Two-dimensional (2D) cell culture remains the primary method of drug screening, despite being considered less physiologically relevant than three-dimensional (3D) culture. Increased complexity and technical challenges of 3D systems have limited its widespread adoption as a primary screening method.

Only 13.8% of drugs entering Phase 1 are ultimately approved by the FDA. (Wong et al., Biostatistics (2019) 20, pp. 273-286). This rate is even lower for oncological drugs: despite strong evidence of their efficacy in laboratory settings with tissue culture models, roughly 96% of drug candidates failed to be both efficacious and safe in humans.

One factor that may be contributing to low approval rate of oncology drugs is the traditional use of two-dimensional (2D) cell culture rather than three-dimensional (3D) culture during drug discovery and screening. 2D cell culture can be faster, less costly, and relatively easier to interpret, compared to 3D. However, there is increasing evidence that 2D culture substantially alters the physiological properties of cells compared to 3D, due to the different microenvironmental cues that exist in both culture methods. 2D culture enforces apical-basal polarity, which is not physiologically relevant for more mesenchymal cell types, and in some circumstances has been shown to alter sensitivity to apoptosis. Two-dimensional culture creates single cell monolayers, which alters diffusion of nutrients, gasses, and drugs, affecting both cell behavior and metabolic activity. (Baker, B. M., et al., 2012 J Cell Sci, 125(Pt 13) pp. 3015-3024). Furthermore, many researchers have demonstrated differences in cell proliferation rates, gene expression, and differentiation in 2D vs. 3D cultures. (Duval, Kayla et al., Physiology 32:266-277, 2017). These deviations from the true in vivo behaviors of cells may lead to Type 1 and Type 2 errors during 2D drug screens, thus ruling in drugs that are clinically futile, and ruling out drugs that would otherwise prove effective. Two-dimensional (2D) cell culture remains the primary method of drug screening, despite being considered less physiologically relevant than three-dimensional (3D) culture. Increased complexity and technical challenges of 3D systems have limited its widespread adoption as a primary screening method.

Cell painting assays for morphological profiling of 2D cell cultures using multiplexed fluorescent dyes have been developed. (Bray, Mark-Anthony et al., Nat Protoc 2016 September 11(9):1757-1774).

Improved methods for morphological profiling, increased throughput, imaging and automation in 3D cell culture assays that are suitable for compound screening using patient-derived samples are desirable. New analysis approaches and descriptors that increase information about disease phenotypes and compound effects are also desirable. There is a critical need to develop methods for efficient testing of drug efficacy in patient-derived tumor samples to discover new therapeutics.

### SUMMARY

Methods are provided for increased throughput, imaging and automation in 3D assays that are suitable for compound screening using patient-derived samples. A Cell Painting method is provided for analysis of 3D tumoroids and evaluation of phenotypic effects.

Methods for phenotypic profiling of three-dimensional (3D) cell biology models are provided. The methods are useful for determining cellular effects in response to exposure to various compounds and environmental conditions. Protocols have been developed and optimized for 3D tissue models such as spheroids and tumoroids including patient-derived tumoroids. Methods are provided comprising obtaining an organoid, treating with a candidate compound, staining with a multiplicity of dyes, imaging the stained tumoroids, and analyzing the images. The organoids may be tumor organoids. The tumor organoids may be obtained from patient derived tumor tissue and subjected to, for example, serial passaging in a mouse xenograft model, or two-dimensional (2D) cell culture, to obtain the 3D tumor organoids. The tumor tissue may be, for example, a breast, brain, bone, adrenal, colon, colorectal, lung, bronchus, bladder, kidney, renal pelvis, liver, intrahepatic bile duct, stomach, oral cavity, oropharyngeal, laryngeal, cervical, melanoma, myeloma, uterine, endometrial, ovarian, pancreatic, prostate, testicular, throat, thymus, thyroid, lymphoma, leukemia, or skin tumor tissue.

Methods for phenotypic profiling of a 3D cell culture are provided comprising obtaining an organoid, treating the organoid with a candidate compound, staining the treated organoid with a multiplicity of dyes, 3D imaging the stained tumoroids, and analyzing the images to provide the phenotypic profile of the 3D cell culture. The 3D cell culture may be selected from the group consisting of an organoid, a spheroid, a tumoroid. The organoid may be a patient-derived organoid.

The phenotypic profiling may be useful in personalized medicine, for example, for selecting an anticancer drug or multiplicity of drugs for use in treating, preventing, or alleviating, a cancer, tumor, or neoplastic condition in a subject in need thereof.

The phenotypic profiling may be useful, for example, for studying various environmental compounds or conditions for preventing, or alleviating, a neoplastic condition, such as, for example, an oncological disease or condition, a cancer, a tumor.

Methods are provided for selecting one or more compounds for treating, alleviating, or preventing a disease or condition in a subject, comprising obtaining a 3D tissue culture model, treating the 3D tissue culture model with one or more candidate compounds, staining the treated 3D tissue culture model with a multiplicity of dyes, imaging the stained 3D tissue culture model, analyzing the images to provide the phenotypic profile of the 3D tissue culture, and selecting one or more of the candidate compounds for treating, alleviating, or preventing a disease or condition based on the phenotypic profile.

The 3D tissue culture model may be selected from the group consisting of an organoid, a spheroid, a tumoroid. The organoid, spheroid or tumoroid may be a patient-derived organoid, spheroid, or tumoroid. The disease or condition may be an oncological disease or condition.

A method of phenotypic characterization of a three-dimensional (3D) target cell culture model is provided, the method comprising culturing the 3D target cell model in wells of a well plate for a first period of time; staining the cultured 3D target cell model with three or more, four or more, five or more, or six or more dyes; imaging the stained 3D target cell model; and analyzing the images to quantify one or more phenotypic characteristics of the 3D target cell model. The imaging may comprise capturing, using a digital imaging device, a first plurality of vertically spaced-apart X,Y images of the wells, each X,Y image having a different height along a Z axis, such that a volumetric image stack is generated with respect to the wells. The respective Z-coordinates of sequential images in the volumetric image stack may differ by at least approximately 1 micrometer, at least 2 micrometers, or at least 3 micrometers. The respective Z-coordinates of sequential images in the volumetric image stack may differ by less than approximately 50 micrometers, less than 40 micrometers, less than 30 micrometers, less than 25 micrometers, or less than 20 micrometers. The respective Z-coordinates of sequential images in the volumetric image stack differ by between 1 micrometer and 25 micrometers, 2 micrometers and 20 micrometers, or 3 micrometers and 15 micrometers.

The imaging may comprise illuminating the stained 3D target cell model with a multiplicity of lasers at different wavelengths suitable for excitation of the fluorescent dyes, optionally wherein images are acquired at different emission wavelengths for each of the fluorescent dyes, and optionally wherein each of the fluorescent dyes are specific for a different phenotypic characteristic.

The method may include monitoring one or more phenotypic characteristics of the 3D target cell model at one or more, two or more, three or more, or four or more time points during the culturing, optionally wherein the monitoring comprises imaging using transmitted light (TL).

The method may include fixing and optionally permeabilizing the cultured 3D target cell model. The method may further include measuring one or more secreted factors in 3D target cell model supernatants. The method may further include treating the cultured 3D target cell model with one or more candidate compounds over a second period of time, optionally wherein the treating is before the staining. The treating may include exposing the cultured 3D target cell model to different doses of the one or more candidate compounds to obtain a dose-response curve for each of the one or more phenotypic characteristics, optionally wherein the different doses include two or more, three or more, four or more, or five or more different doses.

The three or more dyes may comprise at least three dyes each specific for a different biomarker, cellular component or organelle selected from the group consisting of nuclear DNA, lysosomes, RNA, endoplasmic reticulum (ER), nuclei, nucleoli, cytoplasmic RNA, actin, Golgi apparatus, plasma membrane, mitochondria, and cytoskeleton. The three or more dyes may be selected from the group consisting of a cell-permeant cell viability dye, a cell-impermeant dead cell nucleic acid stain, a bis-benzimide DNA stain, a E-cadherin stain, and a cell surface biomarker. The three or more dyes may be selected from the group consisting of fluorescent dyes, luminescent dyes, and quantum dots, optionally wherein the three or more dyes comprise a dye-antibody conjugate, wherein the antibody is capable of specific binding to the selected biomarker, cellular component, or organelle.

The staining may comprise sequentially adding one or more of the three or more dyes to the 3D target cell model. The staining may comprise simultaneously adding the three or more dyes to the 3D target cell model.

The one or more phenotypic characteristics may be selected from the group consisting of 3D target cell model size; diameter; area; disintegration; density; compactness; texture; integrity; optical density; shape; width; cell viability; ATP level; nuclei count; nuclear area; fluorescence intensity; total cell count; live cell count; dead cell count; cell area; projected cell area; number of viable cells; and cell number positive for a selected biomarker, cellular component, or organelle.

The biomarker, cellular component, or organelle may be selected from the group consisting of nuclear DNA, lysosomes, RNA, DNA, endoplasmic reticulum (ER), nuclei, nucleoli, cytoplasmic RNA, actin, Golgi apparatus, plasma membrane, mitochondria, and cytoskeleton.

The 3D target cell model may be selected from spheroid, tumoroid, organoid, and PDX-derived organoids. The 3D target cell model may be derived from a patient tissue, tumor, biopsy sample, or a tumoroid fragment. The method may include obtaining isolating cells from a primary tumor of a patient; cultivating the isolated cells to obtain two-dimensional (2D) cultivated cancer cells or passaging into murine models for expansion to provide xenograft cancer cells; and forming the 3D target cell model from the 2D or xenograft cancer cells.

The method may further comprise monitoring one or more phenotypic characteristics of the 3D target cell model before and at one or more, two or more, three or more, or four or more time points during the culturing and/or treating, optionally wherein the monitoring comprises imaging using transmitted light (TL).

A method of selecting a drug therapy for treatment of a subject in need thereof is provided, the method comprising culturing a 3D target cell model derived from the subject in wells of a well plate over a first period of time; treating the cultured 3D target cell model with one or more candidate drugs over a second period of time; staining the treated 3D target cell model with three or more dyes; imaging the stained 3D target cell model; and analyzing the images to quantify one or more phenotypic characteristics of the treated 3D target cell model.

The method may include calculating a phenotypic distance score for each candidate drug for the one or more phenotypic characteristics compared to an untreated control; and selecting one or more of the candidate drugs having a phenotypic distance score above a threshold value for therapeutic treatment of the subject. The treating may comprise exposing the cultured 3D target cell model to different doses of the one or more candidate drugs to obtain a dose-response curve for each of the one or more phenotypic characteristics, optionally wherein the different doses include two or more, three or more, four or more, or five or more different doses. The analyzing may further comprise clustering the identified candidate compounds based on the phenotypic distance score for one or more phenotypic characteristics to create a phenotypic profile for the 3D target cell model. The method may include calculating the phenotypic distance score for each candidate drug based on the dose-response curve for each of the one or more phenotypic characteristics.

### BRIEF DESCRIPTION OF THE DRAWINGS

The application file contains at least one drawing executed in color. Copies of this patent or patent application publication with color drawing(s) will be provided by the Office upon request and payment of the necessary fee.

FIG. 1 shows a schematic diagram of the experimental workflow including generation of patient-derived cell line, formation of tumoroids in 384 well U-shape low attachment plates, compound treatment, staining, imaging and analysis.

FIG. 2A shows reference images of tumoroids in the following order: tumoroids formed 48 h after plating, TL images (10×)(upper left); composite fluorescent images of untreated tumoroid (upper middle) stained with calcein AM (green), EthD-1 (red) and Hoechst (blue), confocal image, 10×, maximum projection; tumoroids treated with romidepsin (10 nM; upper right), bortezomib (10 nM; middle left), dactinomycin (10 nM; center), plicamycin (10 nM; middle right), carfilzomib (100 nM; lower left), trametinib (100 nM; lower middle), paclitaxel (100 nM; lower right). Organoids were imaged using confocal option of the automated imaging system, Z-stack of 15 images was taken 10 μm apart, then maximum projection images created. (shown).

FIG. 2B shows a 384 well plate with tumoroids after compound treatment, TL images (left); and fragment of test plate with stained tumoroids (right), fluorescent images, 10× magnification.

FIG. 3A shows end-point analysis of fluorescent images of tumoroids done using the Custom Module Editor in MetaXpress High-Content Image Acquisition and Analysis Software. Image analysis was done using the Custom Module Editor for finding organoids, nuclei, and cells. Images of nuclei of Romidepsin treated (right) and untreated organoids (left) shown. Analysis masks show organoids projection in blue and nuclei in yellow. Tumoroid area and count nuclei were used as main read-outs for phenotypic characterization of organoids and compound effects.

FIG. 3B shows automated image analysis of 3D cancer microtissues for control (left), Romidepsin treated (middle), and Trametinib treated (right) was done using transmitted light images (10×) with machine learning-based-based image analysis module In Carta software (analysis mask shown in purple). Further analysis determined the area, optical density, and other read-outs.

FIG. 4 shows images of 3D cancer microtissues treated with indicated selected compounds for 5 days, then stained with calcein AM (green), EthD-1 (red) and Hoechst (blue). Confocal images, 10× shown. Note dose-dependent dis-integration of microtissues, also increase in cell death indicated as increase of EthD-1 staining (in red).

FIG. 5A shows the Cell Painting assay modified for 3D spheroids. Spheroids were labeled with dyes including phalloidin (actin, fuchsia, lower right), MitoTracker (mitochondria, red, lower left), WGA (golgi, orange, upper middle), SYTO 14 (RNAP, yellow, lower middle), concanavalin A (ER, green, upper right) and Hoechst 33342 (nuclei, aqua, upper left). Shown here is an example image of a control spheroid (maximum projection).

FIG. 5B shows a scatterplot representing the phenotypic distance score (−log, Yaxis) for each compound is shown. Compound treated samples (blue) compared to negative controls (red) are shown. Hits are identified as those above the red dotted line (p<0.05).

FIG. 5C shows results from the cluster analysis represented as a hierarchical dendrogram. Left: The cluster ID and colored bars indicate which cluster a compound treated spheroid belongs to. Rows represent the included factors, columns represent the compound treatment. Middle: Correlation matrix is shown to give an overview of the similarity/dissimilarity between the compounds. Columns and rows represent compounds used. The intensity of the color represents the similarity based on the calculated cosine vector score from the PCA factors. Right: Bar graph showing the contribution of each PCA factor to each compound hit.

FIG. 5D shows Spheroids treated with a library of anti-cancer compounds and then stained with the modified Cell Painting assay. Images were acquired using the high content confocal microscopy and analyzed in IN Carta image analysis software. All measurements were then further analyzed using StratoMineR. Shown here are example images of treated spheroids found in the same cluster groups. Three of the stains, Hoeschst (blue, nuclei), SYTO 14 (green, RNAP) and MitoTracker (red, mitochondria) are represented as a composite image. An example of a control DMSO treated spheroid (lower right) and spheroids from cluster 7 (upper left, Temsirolimus-treated or Vorinostat-treated), cluster 6 (upper right; Belinostat-treated orOsimertinib-treated), and cluster 5 (lower left, Encorafenib-treated or Dabrafenid mesylate-treated) are shown.

FIG. 6 shows approximate minimum effective concentrations (in 10-100,000 nm range) shown for active compounds from NCI library.

FIG. 7 shows images of tumoroids that were cultured for 5 days with compounds from NCI library, 10 uM concentration, then imaged on IXM-C HT.ai automated imaging instrument without staining in transmitted light using 10× objective. Images were analyzed using SYNAP module of InCartasoftware (Molecular Devices). Analysis mask shown in yellow. Selected images presented for DMSO controls (D2 and E2), un-effected wells (A7 and A8), also effected by compounds (All, A12 doxorubicin; A17, A18 mitoxantrone, D23, D24 copanlisib; P19, P20 erdafitinib).

FIG. 8 shows a bar graph of lactate secretion in supernatants collected from treated tumoroids following compound treatments. Compound treatments and secretion of lactate were studied using a Pu⋅MASystem, a microfluidic-based automated organoid assay platform that allows multistep protocols. Supernatants were collected from treated tumoroids using flowchipautomation five times after 3 hr incubation periods. The first time point was with media only (baseline); the subsequent four incubations were with media+compound. Tumoroids were exposed to drug at 1 uM concentrations or vehicle, for 12 hours. Supernatants were analyzed for lactate concentration using the Lactate-Glo assay (Promega).

FIGS. 9A-9D show Table 5 with data from analysis of tumoroid images after 5 days of treatment with 10 uM concentrations of indicated compounds. The well, area, diameter, average intensity, total intensity, intensity CV, light flux, and background intensity are shown for each compound. Images were taken after staining cells as indicated in methods section. Images were analyzed using MetaExpress software. Numbers represent nuclei counts in the projection images of the tumoroids. Numbers were conditionally formatted if they were above (green) or below (red) of 2STDEV from the DMSO controls for each concentration separately. Data from highest concentration is grayed out as less reliable since high concentrations of compounds caused severe cell damage and potential errors in nuclear count.

FIGS. 10A-10G show Table 6 with data including well, area, diameter, perimeter of objects, average intensity, total intensity, intensity CV, light flux, and background intensity from analysis of tumoroid images after 5 days of treatment with 10 uM concentrations of indicated compounds. Images were taken with transmitted light, 10× objective, then analyzed using InCarta software. Synap module of InCarta software was created to find tumoroids, then the objects were measured for dimensions and intensity.

### DETAILED DESCRIPTION OF THE INVENTION

The current state of the drug development pipeline reflects the need for better research models. According to estimates of clinical trial success rates, only 13.8% of drugs entering Phase 1 are ultimately approved by the FDA. This rate is even lower for oncological drugs, with an average rate of approval of 3.4% per year, based on data from 2000-2015.

In the context of cancer, there is increasing evidence that 2D culture is insufficient to evaluate the effects of drugs. In breast cancer specifically, there is evidence that culturing methods affect response to drug treatment. El-Feky et al., 2021, Exp Ther Med 21(5), 506 found that MCF7 cells cultured in 2D monolayers and 3D spheroids had differential response to FACbased chemotherapy (5-fluorouracil, Adriamycin (doxorubicin), cyclophosphamide), with 2D cultures showing a greater reduction in viability. Additionally, Imamura et al., 2015, Oncology reports, 33(4), 1837-1843, demonstrated that 3D spheroids of breast cancer cell lines (BT-549, BT-474 and T-47D) had greater resistance to doxorubicin and paclitaxel compared to 2D. However, these studies were only able to examine a limited number of chemotherapeutic agents, at limited concentrations, thus demonstrating the need for more robust, high-throughput drug screens using 3D models of breast cancer.

New advanced models that include primary derived tumoroids and 3D cultures present significant technical challenges that have prevented their adoption as the predominant methods of cell culture for drug discovery and screening. 3D micro-tissues take longer to form, they are more heterogeneous and fragile, and they are more complicated to image and analyze, all of which increases the time and complexity required to perform experiments. The increased complexity of cell models requires the development of new methods that overcome these challenges and provide the best information from that complexity. It is also extremely important to develop new models for specific cancers that are derived from primary patient tumors. The use of patient-derived tumor tissues has transformed the field of drug and target discovery research, providing a translational tool and physiologically relevant system to evaluate tumor biology. An example of this method is the use of patient-derived organoids (PDO) for oncology research (www.fda.gov/drugs/new-drugs-fda-cdersnew-molecular-entities-and-new-therapeutic-biological-products/novel-drug-approvals-2021; Ishiguro et al., 2017, Cancer Sci, 108(3), 283-289; Jensen and Teng, 2020, Frontiers in molecular biosciences, 7, 33; Matossian et al., 2021, Clinical & translational oncology, 10.1007/s12094-021-02677-8.)

Patient-derived tumor organoids exhibit the heterogeneity of tumor tissues and presence of cancer stem cells (CSC) that can be expanded over multiple passages to produce large numbers of tumoroids (derived from isolated cells) or organoids (derived from digested tumors), that maintain molecular characteristics of the original tumor (Jensen and Teng, 2020; Matossian et al., 2021, ibid.). Primary-derived cell lines represent a variety of cell subtypes present in the tumor, as well as the different mutations involved and the degree of malignant transformation. Development of various cell lines for breast cancer, or other cancers, that represent disease subtypes is critical for finding drugs or drug combinations that would be effective against that specific cancer, or even for that specific patient. Study and characterization of patient derived tumor cells is an active area of investigation, and importance is increasing with the need to develop patient-specific therapies. However, drug testing in patient-derived tumor cells is not widely adopted because of the additional technical challenges, related to difficulties in expansion, handling, and maintenance of primary-derived tumoroids. In this study, a patient-derived 3D cell model was used representing a rare drug-resistant breast cancer subtype, which allows us to model the cellular and molecular complexity and diversity of breast cancer. In addition, a variety of methods for automation and high-content analysis adapted for the culture and drug screening of primary tissue-derived 3D cell models are presented. A library of approved anti-cancer drugs was tested and defined several potentially promising candidates for treatment of this specific cancer type.

Methods for measuring responses to drug treatment in 3D tumoroids for cytotoxicity and altered morphology are provided herein. In some methods, tumoroids are formed from primary cells isolated from a patient-derived tumor explant, and treated with one or more, two or more, three or more, or a library of candidate compounds, optionally at multiple concentrations. The treated 3D tumoroids and optionally supernatants thereof are analyzed by various techniques to obtain values for a multiplicity of quantitative descriptors for tumor phenotypes and compound effects.

For example, as provided herein, tumoroids were formed from primary cells isolated from a patient-derived tumor explant, TU-BcX-4IC, that represents metaplastic breast cancer with a triple-negative subtype, and treated with 165 compounds different approved cancer drugs, at multiple concentrations. Multiple quantitative descriptors for tumor phenotypes and compound effects were characterized. For example, a Cell Painting method was used for 3D tumoroids for evaluation of phenotypic effects. Eight compounds were identified that demonstrated effects at low concentrations (10 nM), including romidepsin, trametinib, bortezomib, carfilzomib, panobinostat, which were further investigated as potential drug candidates.

In the present disclosure, a patient-derived cell line was used representing a rare drug resistant cancer subtype for drug screening. The results of the library testing of approved anti-cancer drugs are presented that were tested at different compound concentrations, using high content imaging methods. Methods are described for increase of throughput by using automation in 3D cancer assays and compound screening. In addition, advanced analysis approaches and descriptors are shown that allow for greater information about complex compound effects.

A key strength of the present method development is the usage of a primary tissue-derived cell model. The standard approach is to use the established immortalized cell lines as well as orthotopic xenograft models. (Langhans 2018, Front. Pharmacol. 9:6. doi: 10.3389/fphar.2018.00006; Prieto-Vila et al., 2017, Int. J. Mol. Sci. 18:E2574; Nguyen et al. 2018, Molecular cancer therapeutics, 17(12), 2689-2701; Zhang et al., 2020, J Tissue Engineering 11:1-17).

Although immortalized cellular models provide invaluable knowledge regarding cancer biology and drug effects on cellular systems, they are limited in their inability to re-create essential features of tumors. More specifically, these models cannot accurately reflect the tumor architecture, three-dimensional structure and alignment of tumor cells, matrix, and surrounding stroma, and cannot reproduce the cellular heterogeneity that is present in the original patient tumor. (Burdall et al., 2003; 5:89-95; Manning et al., 2016, J Nuclear Med; 57(S1):60S-68S; Gillet et al., 2011; Proc Natl Acad Sci USA; 108:18709-13).

Conversely, orthotopic xenograft models recapitulate the complexity of tumors, but the inability to scale these models limits their use in large drug screens. The methods described in this paper use primary cell-derived models to form 3D spheroids, achieving greater relevance of the results to real biology and allowing for large drug screens to be performed.

Beyond the use of patient derived cells, using 3D cultures rather than 2D cultures for screening allows closer recapitulation of properties of tumors. Cells in 3D spheroids are in close contact with each other, as they would in a human body (Langhans 2018, Front. Pharmacol. 9:6. doi: 10.3389). Also, cells in dense, multicellular micro-tissues have a higher rate of hypoxia compared to 2D monolayers, which has been shown to be associated with drug resistance. (Imamura et al., 2015, Oncology reports, 33(4), 1837-1843; Rajcevic et al., 2014, Proteome science, 12, 39).

Drug penetration into the tissue also plays role in drug efficacy, and that factor can be mimicked during screens in 3D models. In general, drug screening and discovery may result in two types of errors: type 1, false positive drugs, or type 2, false negative errors that can ultimately result in loss of drugs that may have proven clinically effective.

Using more biologically relevant models would decrease both false positive and false negative types of errors. Immortalized cell lines that have been growing in cell culture for years results in the introduction of irreversible alterations in genetic information and behavioral characteristics that were not present in the original tumor. (Gillet et al., Proc Natl Acad Sci USA, 2011; 108:18709-13). As a result, those cell lines would be most responsive to anti-proliferative drugs and may not be affected by other drug classes. Cells cultured in 2D have altered morphology and organization of cell surface receptors compared to 3D, which could affect the binding efficacy of drugs and their penetration inside the tumor. (Jensen et al., 2020, Frontiers in molecular biosciences).

In the present disclosure, several assays have been developed suitable for medium- or large-scale compound testing. Various analytical methods allowed us to evaluate different aspects of compound effects: tumoroid integrity that is evaluated by measurement of spheroid area or cell count, cytotoxic effects that were evaluated by livedead stain, and spheroid integrity that was evaluated with just nuclear stain. As expected, a progressive increase was observed in the number of effective compounds with the increase of concentration. Eight compounds were effective at a 10 nM concentration, which demonstrated high efficacy and potentially can be considered for drug development. Several of those compounds were kinase inhibitors, which is a promising drug class for cancers resistant to traditional anti-cancer therapies. Interestingly, cell disintegration appeared as an effective read-out at lowest concentration, while the actual increase of dead cells appeared at higher concentrations for the same drugs. That may indicate that loss of adhesion molecules may be occurring, or that subtle changes in viability have resulted in loss of cell-cell attachment. The Cell Painting method is of particular interest due to it unbiased approach, which captures phenotypic changes, independent of cytotoxicity. Information from multiple stains that label various organelles and cellular compartments are used to represent the morphological profile of each cell. For 3D cell painting, due to the proximity of cells to each other, it is challenging to analyze the data at the cellular level. Here, we examine the effects of compounds on the phenotypic profile at the spheroid level. Interestingly, spheroid-level analysis identified not only cytotoxic compounds but also compounds that have significant phenotypic effects.

Cluster analysis grouped compounds with similar mechanisms of action, suggesting that spheroid-level analysis is sufficient for assessing compound effects. While Cell Painting and toxicity analysis showed the same hits for strong compounds, compounds with more subtle effects showed different results between the two assays, reflecting toxicity vs. other phenotypic changes. There is an unmet need for greater accuracy in drug screening for cancer, especially in the expanding area of personalized medicine. Approaches are disclosed that can overcome technical challenges and make feasible drug screening that would be suitable for specific cancer subtypes or even individual patients. Through automation, large-scale drug screening using complex models, including tumoroids derived from individual patients, is feasible. In the future, this method would be suitable for screening other cancer subtypes that form tumoroids, such as colon and other cancers. Additionally, through the greater biological relevance and high-throughput nature of this system, other compound libraries can be screened.

### Definitions

The terminology used herein is for the purpose of describing particular embodiments only and is not intended to be limiting of the disclosure.

The singular forms “a”, “an” and “the” are intended to include the plural forms as well, unless the context clearly indicates otherwise.

The term “and/or” refers to and encompasses any and all possible combinations of one or more of the associated listed items.

The term “about,” when referring to a measurable value such as an amount of a compound, dose, time, temperature, and the like, is meant to encompass variations of 10%, 5%, 1%, 0.5%, or even 0.1% of the specified amount.

The term “antibody conjugate” refers to a specific antibody conjugated to a chromophore or fluorophore molecule, an avidin such as a streptavidin, or a biotin, wherein the antibody is specific for a target antigen such as a secreted factor.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

All patents, patent applications and publications referred to herein are incorporated by reference in their entirety.

The embodiments described in one aspect of the present disclosure are not limited to the aspect described. The embodiments may also be applied to a different aspect of the disclosure as long as the embodiments do not prevent these aspects of the disclosure from operating for its intended purpose.

The terms, “patient”, “subject” or “subjects” include but are not limited to humans, the term may also encompass other mammals, or domestic or exotic animals, for example, dogs, cats, ferrets, rabbits, pigs, horses, cattle, birds, or reptiles.

The term “therapeutically effective amount” refers to the amount of a compound that, when administered to a subject for treating a disease or condition, is sufficient to effect such treatment for the disease or condition. The “therapeutically effective amount” can vary depending on the compound, the disease and its severity, and the condition, age, weight, gender etc. of the subject to be treated.

“Treating” or “treatment” of a disease state or condition includes: (i) preventing the disease state or condition, i.e., causing the clinical symptoms of the disease state or condition not to develop in a subject that may be exposed to or predisposed to the disease state or condition, but does not yet experience or display symptoms of the disease state or condition, (ii) inhibiting the disease state or condition, i.e., arresting the development of the disease state or condition or its clinical symptoms, or (iii) relieving the disease state or condition, i.e., causing temporary or permanent regression of the disease state or condition or its clinical symptoms.

The term “target cells” refers to cells for automated cell culture applications of the present disclosure, such as organoids, tumoroids, spheroids, stem cells, or a production cell line. In some embodiments, the target cells are spheroids, tumoroids, organoids and/or other multi-cellular bodies. In some embodiments, the target cells may be stem cells. In some embodiments, the target cells may be a production cell line. The target cells may be derived from a target tissue. The target tissue may be a mammalian primary tissue, or an organoid, or tumoroid. The mammalian tissue may be derived from a patient biopsy sample. The target tissue may be derived from target organs such as, e.g., lung, intestine such as small intestine, colon, stomach, pancreas, liver, kidney, skin, bone marrow, blood-brain barrier, brain, heart, and the like.

The term “three-dimensional (3D) target cell culture model” or “3D target cell model” refers to a selected 3D multicellular in vitro tissue culture aggregate composed of one or more cell types. The 3D target cell model may be selected from, for example, a spheroid, tumoroid, organoid, or PDX-organoid, and the like. The 3D target cell model may be derived from a patient tissue sample.

Organoid, spheroid, tumoroid, and three dimensional (3D) cell culture models are useful in many applications such as disease modeling and regenerative medicine. 3D cellular models like organoids and spheroids may be useful to better understand complex biology in a physiologically relevant context because cells often retain natural shape and proper spatial orientation, such as in aggregates or spheroids, whereas 2D models of cells grown in a sheet or monolayer may not be as successful. Gene and protein expression of 3D cell culture may more closely mimic gene and protein expression. For example, 3D cell cultures may be useful for drug target identification, lead compound identification, compound optimization, preclinical attesting, solid tumor modeling, genetic disease modeling, drug discovery, precision medicine, organs-on-chips, and bioprinting.

The term “spheroids” refer to three dimensional (3D) multicellular in vitro tissue cultures aggregates composed of one or more cells types that grow and proliferate, and may exhibit enhance physiological responses, but do not undergo differentiation or self-organization. Common cell sources for spheroids are primary tissues or immortalized cell lines. Spheroids may bridge the gap between monolayers and complex organs.

The term “tumoroid” refers to three dimensional (3D) multicellular in vitro tissue culture aggregates composed of one or more cell types typically derived from primary tumors harvested from oncological patients. Tumoroids can mimic human tumor microenvironment in at least one aspect. Tumoroids may be useful for studies on novel cancer drugs or for use in precision medicine in the field of oncology. Cancer cells or cancer cell lines may be, for example, breast, bladder, colon, hematopoietic, lymphoid, liver, lung, ovary, prostate, brain, skin cancers, and the like.

The term “organoids” refer to three dimensional (3D) multicellular in vitro tissue culture aggregates composed of one or more cell types, in which cells spontaneously self-organize into properly differentiated functional cell types and progenitors that resemble their in vivo counterparts in at least one aspect. Organoids mimic their corresponding in vivo organs. Organoids can be derived from pluripotent stem cells (PSCs), induced pluripotent stem cells (iPSCs), neonatal tissue stem cells, embryonic stem cells (ESCs), adult stem cells, or primary tissue. Organoid cultures can be crafted to resemble much of the complexity of an organ, therefore are useful for study of disease etiology and treatment. Organoid technology has recently emerged as an essential tool for both fundamental and biomedical research. The organoid cultures may be selected from different types of target organs such as, e.g., lung, intestine such as small intestine, colon, stomach, pancreas, liver, kidney, skin, bone marrow, blood-brain barrier, brain, heart, and the like.

The term “stem cells” refers to undifferentiated cells that have the potential to develop into many different cell types that carry out different functions. Pluripotent stem cells, such as those found in embryos, can give rise to any type of cell such as those in brain, bone, heart, and skin. Some human adult cells can be reprogrammed into embryonic stem cell-like state called induced pluripotent stem cells (iPSCs). Multipotent stem cells, for example, found in adults or in babies umbilical cords, may develop into the cells that make up the organ system that they originated from. When grown under certain cell culture conditions, pluripotent stem cells can remain undifferentiated. To generate differentiated cells, the chemical composition of the culture medium may be changed, the surface of the culture dish may be altered, or the cells may be modified by forcing expression of certain genes.

The term “PDX-derived organoids” (PDXOs) refers to 3D in vitro models generated from patient tumor tissue that has been previously passaged into murine models for expansion. PDXO are in vitro models derived from in vivo patient-derived xenografts (PDX).

The term “production cell line” refers to a cell line that can be used for production of enzymes, vaccines, monoclonal antibodies, cytokines, peptides, therapeutic toxins, clotting factors, Fc-fusion proteins, or hormones, and the like. The production cell line can be a prokaryotic or eukaryotic production cell line. The production cell line may be any production cell line appropriate for protein or virus production. Prokaryotic production cell lines may include any appropriate prokaryotic cell line. The prokaryotic production cell lines may include any appropriate bacterial cell line (e.g., Escherichia coli). Eukaryotic production cell lines may include Chine Hamster Ovary (CHO) cell lines, murine myeloma cell lines (e.g., NS0, Sp2/0), murine C127, human embryonic kidney (HEK) cell line such as a HEK293 cell line, an human fibrosarcoma cell line such as a HT-1080 cell line, human embryonic retinal cell lines such as a PER.C6 cell line, a baby hamster kidneys (BHK) cell line such as a BHK21 cell line, yeast cell lines (e.g., Saccharomyces cerevisiae, Pichia pastoris), insect cell lines infected with viral vector baculovirus (baculovirus-insect cell expression system), e.g., a Sf9 cell line, plant cells.

The term “feeder cells” refers to cells which provide extracellular secretions to help another cell to proliferate, grow, differentiate, and/or maintain identity. Feeder cells may support growth of target cells in culture by contributing a complex mixture of extracellular matrix (ECM) components and growth factors. In some cases, the feeder cells may be unable to divide, i.e., have arrested cell growth. Feeder cell growth may be arrested by, for example, any appropriate methods known in the art. Feeder cell growth may be arrested by chemical fixation, for example, by mitomycin-C or glutaraldehyde chemical fixation. Feeder cell growth may be arrested by physical methods, for example, gamma irradiation, x-ray irradiation, or electric pulses. For example, feeder cells used for co-culture of target cells such as embryonic stem cells (ESCs) may be fibroblasts which may be mitotically inactivated so they remain viable. In some cases, target cells may be grown in the presence of feeder cells capable of dividing. Some live feeder cells (such as human fibroblasts) may also become target cells as in the case of induced pluripotent stem cells (iPSCs) upon reprogramming. Feeder cells may be arrested feeder cells, for example, that are unable to divide. Feeder cell selection may be dependent on target cells. Feeder cells may be, for example, fibroblasts that are not arrested. Feeder cells may be arrested fibroblasts, epithelial cells, mesenchymal cells, muscle cells, stromal cells, spleen cells, or amniocytes. The fibroblasts may be, for example, human dermal fibroblasts, 3T3 fibroblast cells, human fetal fibroblasts, mouse embryonic fibroblasts, and the like. The epithelial cells may be, for example, human adult fallopian tubal epithelial cells, human amniotic epithelial cells, HeLa cells (human cervical cancer carcinoma epithelial cells), and the like. The mesenchymal cells may be adipose-derived mesenchymal stem cells, human bone marrow-derived mesenchymal stem cells, human bone marrow-derived mesenchymal cells, human amniotic mesenchymal stem cells, and the like. The stromal cells may be, for example, human bone marrow stromal cells, or mouse bone marrow stromal cells. The amniocytes may be, for example, human amniocytes or mouse amniocytes.

The term “secreted factor” or “secreted factors” refers to factors that may be secreted from the 3D target cell model during the automated cell culture applications of the present disclosure. The secreted factors may be any secreted factor including hormones, metabolites, enzymes, vaccines, monoclonal antibodies, cytokines, peptides, therapeutic toxins, clotting factors, or Fc-fusion proteins.

Different media components may be required for each type of source cells used, and the type of differentiation to be achieved. The media may be a liquid media. The media may be a complex media or synthetic media. The media may be a complete media. The media may be a semi-solid media. The media may include a hydrogel. The media may be an undefined media or a defined media. The media may be a serum free media. The media may include a serum. The serum may be FBS. The media may include an isolated or synthetic albumin. The media may include, for example, a defined media with added nutrients, amino acids, hormones, and/or growth factors. The amino acids may include essential amino acids. The amino acids may include non-essential amino acids. The media may be a Minimal Essential Medium (MEM) or a Dulbecco's Modified Eagle Medium (DMEM), Advanced DMEM, and the like. The medium may be supplemented with one or more nutrients, growth factors. Tumoroids may be cultured with, for example advanced DMEM with glucose, non-essential amino acids (NEAA), 2 mM glutamine and insulin 120 μg/L, 10% FBS (Gibco 12491-015). For metabolic assays, tumoroids may be cultured with DMEM+10% dialyzed serum (2 mM glutamine, 5 mM glucose, without phenol red).

The media may comprise growth factors. Growth factors such as EGF, Noggin (NOG), R-spondin (RSPO1), HGF, BMP, FGF, and the like may be added to the media. The growth factors may be generated by the feeder cells. The growth factors may be recombinant growth factors. The recombinant growth factor proteins for organoid culture may include, for example, recombinant human EGF protein, recombinant HGF proteins such as, for example, human HGF protein, cynomolgus HGF protein, human FGF10, human Noggin/NOG protein, human RSPO1 protein, human BMP-2 protein, and the like. Additional recombinant growth factors for organoid culture may include, for example, EGF, FGF2, FGF7, FGF9, FGF10, HGF, NOG, RSPO1, RSPO3, Activin A, BMP2, and BMP4, and the like. The tissue culture media, or recombinant growth factor proteins for organoid culture, may be commercially available from, for example, Sino Biological, Inc., or Thermo Fisher Scientific.

In some embodiments, 3D cellular models like organoids and spheroids may be cultivated in a tissue culture media comprising a hydrogel, such as in a hydrogel dome within the media.

The term “hydrogel” or “hydrogels” refers to an extracellular matrix useful for culturing organoids. The hydrogel may include murine EHS sarcoma matrix, for example, available commercially as Matrigel (Corning), Cultex (Trevigen), Geltrex (Gibco), collagen type I, fibrin, hyaluronic acid (HA), gelatin methacrylate (GelMA), decellularized matrices, or biopolymers such as alginate, silk, nanocellulose; engineered materials such as polyethylene glycol (PEG), self assembling peptides such as RADA16/PuraMatrix bQ13, poly(lactic/(co)glycolic) acid, polycaprolactone, polyacrylamide, oligo(ethylene glycol)-substituted polyisocyanopeptides, ELP (elastin-like protein), or combinations of these polymers.

### Dyes

The disclosure provides methods for morphological profiling of 3D target cells. After culturing the 3D target cells and treating the 3D target cells to one or more candidate compounds, the treated 3D target cells may be stained with a multiplicity of dyes. The multiplicity of dyes may include two or more, three or more, four or more, five or more, six or more, or seven or more dyes. The dyes may be conjugated to a specific antibody or other specific antigen recognition molecule specific for a cell type, biomarker, cell surface marker, a cellular component, an organelle, or an excreted factor. The cellular component or organelle may be, for example, nuclear DNA, lysosomes, RNA, endoplasmic reticulum (ER), nuclei, nucleoli, cytoplasmic RNA, actin, Golgi apparatus, plasma membrane, mitochondria, and the like. The dyes may include an antibody conjugate. For example, the dye may be conjugated to an antibody specific for a cell type, cellular component, organelle, protein, enzyme, or a secreted factor. For example, the biomarker may be a CD antigen cell surface biomarker. The CD biomarker may be used for immunophenotyping of a cell. The presence or absence of a specific antigen from the surface of a particular cell population ay be denoted as “+” or “−”, respectively. A pattern of one or more cell surface biomarkers may be used to identify cell type, e.g., stem cells CD34+; all leukocytes CD45+; granulocytes CD45+, CD15+; monocytes CD45+, CD14+; T-lymphocytes CD45+, CD3+; B-lymphocytes CD45+, CD19+; thrombocytes CD45+, CD61+; helper T-cells, CD45+, CD4+, CD3+; regulatory T-cells CD45+, CD25+; cytotoxic T-cell CD45+, CD3+, CD8+; natural killer cells CD56+, CD3−; and the like.

The dye or dye-antibody conjugate may be specific for nucleic acids, DNA, RNA, nuclear DNA, cytoplasmic RNA, endoplasmic reticulum (ER), lysosomes, nuclei, nucleoli, actin, Golgi apparatus, plasma membrane, mitochondria, plasma membrane, cytoskeleton, cell membrane, SC35 (non-snRNP small nuclear ribonucleoprotein particles), anillin, alpha-tubulin, phosphor-p38, phosphor-extracellular signal-regulated kinase (ERK), p53, c-Fos, phosphor-adenosine-3′,5′-monophosphate response element-binding protein (CREB), calmodulin, and the like. The dye may be cell permeant or cell membrane-impermeant. In some cases, the dyes may include a cell-permeant cell viability dye (e.g., calcein AM), a cell-impermeant dead cell nucleic acid stain (e.g., EthD-III), a bis-benzimide DNA stain (e.g., Hoescht 33342), a E-cadherin stain (e.g., Cell Signaling Technology E-cadherin rabbit mAb 3195), and/or a cluster of differentiation (CD) cell surface biomarker, e.g., CD44 stain (e.g., PE-anti-human CD44 Ab, BioLegend 338807).

The dyes may include, but are not limited to, for example, phalloidin (actin), MitoTracker (mitochondria), WGA (golgi), SYTO 14 (RNAP), concanavalin A (ER), Hoechst 33342 (nuclei), calcein, calcein AM, EthD-I, EthD-III, DAPI, FITC, YFP, Texas Red, a cycaine dye such as Cy5, Cy7, and the like.

Dyes specific for mitochondria are commercially available, for example, from Invitrogen or ThermoFisher Scientific. Dyes specific for mitochondria may include green-fluorescent probes such as, for example, nonyl acridine orange, DiOC6(3) (3,3′-dihexyloxacarbocyanine iodide), DiOC7(3), MitoTracker™ Green FM, Rhodamine 123, SYTO 18 yeast mitochondrial stain, JC-1 dye mitochondrial membrane potential probe, JC-9 dye mitochondrial membrane potential probe, dihydrorhodamine 123, and the like. Probes for mitochondria may include yellow- and orange-fluorescent probes, such as, for example, Mito Tracker orange CMTMRos, Rhodamine 6G, tetramethyl rhodamine methyl ester (TMRM), tetraethyl rhodamine ethyl ester, and the like. Probes for mitochondria may include red-fluorescent probes, such as, for example, Mito Tracker™ Red CMXRos, JC-1 dye mitochondrial membrane potential probe, JC-9 dye mitochondrial membrane potential probe, Mito Tracker™ Red FM, Mito Tracker™ Deep Red FM, and the like. Oxidation sensitive probes for mitochondria include dihydrorhodamine 123, Mito Tracker Orange CM-H2TMRos, and MitoTracker™ Red CM-H2XRos, and the like.

Dyes specific for acidic organelles such as lysosomes include Lyso Tracker Green DND-26, LysoTracker Yellow HCK-123, Lyso Tracker Red DND-99, hydroxystilbamidine, LysoTracker Deep Red, Lyso Tracker Blue DND-22, RedoxSensor Red CC-1, and the like.

Dyes that are pH-sensitive probes for acidic organelles include LysoSensor Green DND-189, Acridiene orange, LysoSensor Yellow/Blue DND-160, LysoSensor Yellow/Blue 10,000 MW dextran, Neutral Red, LysoSensorBlue DND-167, LysoSensor Yellow/Blue 10,000 MW dextran, and the like.

Dyes specific for the endoplasmic reticulum include ER-Tracker Green, DiOC6(3), DilC18(3), DilC16(3), Rhodamine B hexyl ester, ER-Tracker Red, ER-Tracker Blue-White DPX, concanavalin A, and the like.

Dyes specific for the Golgi Apparatus may include BODIPY FL C5-ceramide, NBD C6-ceramide, BOPIPY FL C5-ceramide, BODIPY TR ceramide, WGA, and the like.

The term “allophycocyanin” or “APC” refers to a dye isolated from the phycobiliprotein family isolated from algae. It is excitable by laser lines at 594 and 633 nm, with emission at ˜660 nm.

The term “calcein AM” refers to calcein acetoxymethyl ester which is a cell-permeant dye that can be used to determine cell viability in most eukaryotic cells. In live cells, the non-fluorescent calcein AM is converted to green-fluorescent calcein after acetoxymethyl ester hydrolysis by intracellular esterases. Calcein AM positive cells indicates viable cells. Calcein has excitation/emission wavelengths of 495/515 nm respectively. Calcein AM is commercially available from, for example, BioLegend, San Diego, CA.

The term “EthD-III” refers to ethidium homodimer III which is a red fluorescent dead cell stain for bacteria and mammalian cells. It is a cell membrane-impermeant nucleic acid dye that stains only dead cells with damaged cell membranes. EthD-III positive cells indicates dead cells. EthD-III has an excitation max ˜522 nm and emission max ˜593 nm. EthD-III is commercially available from Biotium or ThermoFisher Scientific.

The term “EthD-I” refers to cell impermeant viability indicator ethidium homodimer-I which is a high-affinity nucleic acid stain. EthD-I is weakly fluorescent until bound to DNA and emits red fluorescence having an excitation max of ˜528 nm and an emission max of ˜617 nm. EthD-I binds to both DNA and RNA in a sequence-independent manner with ˜30-fold fluorescence enhancement. Since it cannot penetrate living cells EthD-I is generally used to detect dead cells. EthD-I is commercially available from Biotium or ThermoFisher Scientific.

The term “Hoechst 33342” or “Hoechst” refers to a benzimidazole fluorescent DNA stain that binds within the minor groove of double-stranded (ds) AT-rich regions. The stain can be used on live or fixed cells. The free dye excitation max is ˜340 nm, while the DNA complex excitation max is ˜355 nm. The free dye emission max is ˜510 nm, the DNA complex emission max is ˜465 nm. Hoechst 33342 is commercially available, for example, from Life Technologies, Carlsbad, CA.

The term “DAPI” refers to 4′,6-diamidino-2-phenylindole which is a blue fluorescent DNA stain that exhibits ˜20 fold enhancement of fluorescence upon binding to AT regions of dsDNA. It is excited by the violet (405 nm) laser line and commonly used as a nuclear counterstain in fluorescence microscopy, flow cytometry, and chromosome staining. DAPI emission max is in a range of ˜452 nm to ˜470 nm. DAPI is commercially available from ThermoFisher Scientific.

The term “FITC” refers to fluorescein isothiocyanate (e.g., fluorescein 5-isothiocyanate) which is a green fluorophore that may be excited with a 488 nm laser line and emission max ˜520 nm. FITC may be sensitive to pH changes. FITC is commercially available from several suppliers such as Invitrogen, ThermoFisher Scientific.

The term “Syto14” refers to a cell-permeant green fluorescent nucleic acid dye that exhibits green fluorescence upon binding to nucleic acids. Syto14 has an excitation wavelength range of 517, 521 nm and emission at 549, 547 nm. SYTO™14 is commercially available from Invitrogen., Thermo Fisher Scientific.

The term “YFP” refers to yellow fluorescent protein which may be isolated from jellyfish Aequorea Victoria and has an excitation peak at ˜513 nm and emission peak ˜527 nm and emission max ˜562 nm. YFP analogs may include Citrine, Venus and Ypet, having amino acid substitutions which may reduce chloride sensitivity, increased brightness (product of extinction coefficient and quantum yield).

The term “Texas Red” refers to a red fluorescent dye which may be excited by 561 or 594 nm laser lines and emission max ˜624 nm. Texas Red, or Texas Red-X are commercially available, for example, from ThermoFisher Scientific and may be used to label protein conjugates such as phalloidins for cytoskeleton counterstaining or antibody conjugates for immunofluorescence.

The term “phycoerythrin” or “PE” refers to a red protein fluorescent dye belonging to the phycobiliprotein family. PE can be conjugated to antibodies for antigen detection.

The dye may be a cyanine dye. Cyanine dyes may be abbreviated as Cy3, Cy5, Cy7 and the like.

The term “Cy5” or “Cy5 dye” refers to cyanine 5 which is a far-red fluorescent dye which is excitable by the 633 nm or 647 nm laser lines and emission max ˜692 nm. Various Cy5 dyes and Cy5 derivatives are commercially available, for example, from ThermoFisher Scientific.

The term “Cy7” or “Cy7 dye” refers to cyanine 7 dye excitable by the 633 nm or 647 nm laser line and having a far red or near IR emission maximum ˜775 nm-˜794 nm. Various Cy dyes are commercially available.

The term “WGA” refers to a wheat germ agglutinin dye specific for Golgi apparatus

The term “triple negative breast cancer” is any breast cancer that lacks or shows low levels of estrogen receptor (ER), progesterone receptor (PR), and human epithelial growth factor receptor 2 (HER2) overexpression and/or gene amplification (i.e., the tumor is negative on all three tests ER, PR, and HER2).

The term “principal component analysis” (PCA) refers to a statistical technique for reducing the dimensionality of a data set by transforming a large set of variables into a smaller one that still contains most of the information of the large set. The data may be linearly transformed into a new coordinate system where the variation of the data can be described with fewer dimensions than the initial data. The first two principal components may be employed in order to plot the data in two dimensions and to visually identify clusters of closely related data points. The principal component analysis algorithm may first find the direction with the largest variance in the data set and mark the direction as component 1.

The term “phenotypic distance score” refers to a measure of the phenotypic effect of the compounds on the 3D target cell model relative to the control. The phenotypic distance score may be calculated based on principal component analysis (PCA) components. For profiling studies, it may be useful to reduce each population of descriptor values to a single number. See, for example, Perlman et al., 2004, Science, 306, 1194-1198, suggesting a measure based on Kolmogorov-Smirnov (KS) statistic, allowing non-parametric comparison of experimental and control distributions from the same plate. Dividing by a measure of the variability within a control population may yield a z score, which can be displayed as a function of descriptor and drug concentration in a heat plot to allow rapid visual comparison of response profiles. The plots may represent a family of dose-response curves for a single drug but may differ from traditional curves reflecting changes in in a biochemical measurement. The relationship between the z score and original physical measure may be nonlinear.

Imaging may comprise transmitted light (TL) imaging, fluorescent (FL) imaging, and/or confocal fluorescent imaging.

Measurements can be done with a microscope or with a plate reader. A plate reader has the advantage of allowing a high dynamic range but may require about 1 mm² for every detection array point. An imager can detect a higher number of array points being able to observe much smaller array points but may have a more limited dynamic range.

Cell models for compound screening methods may be developed that that use primary tissue-derived tumoroids. Tumoroids may be formed from any appropriate cell line derived from primary tumor samples. In the present examples, a primary tissue-derived cell line, TU-BcX-4IC, was prepared as previously described. Briefly, patient-derived tumor samples may be implanted into SCID mice, serially passaged, and then expanded in 2D culture (Matossian, et al., 2021). TU-BcX-4IC represents metaplastic breast cancer with a triple-negative breast cancer (TNBC) subtype and is an example of a highly heterogeneous phenotype of breast cancer. TNBC tumors have an aggressive clinical presentation due to high rates of metastasis, recurrence and chemoresistance. The original patient's tumor for the TU-BcX-4IC model exhibited rapid pre-operative growth despite conventional combination therapy with Adriamycin (doxorubicin), cyclophosphamide and paclitaxel. Tumoroids were formed from 2D TU-BcX-4IC cells as described in Methods.

Development and Optimization of the Live Cell High-Content Assay with 3D Spheroid Cultures.

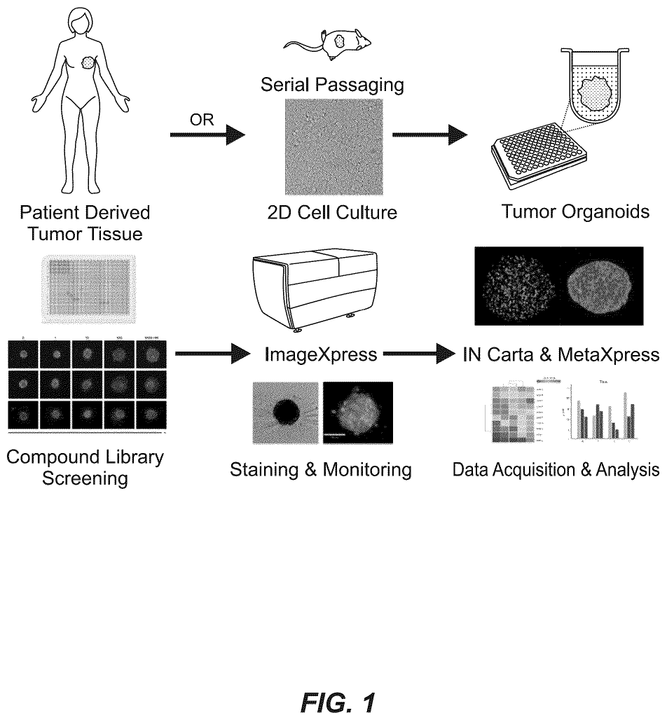

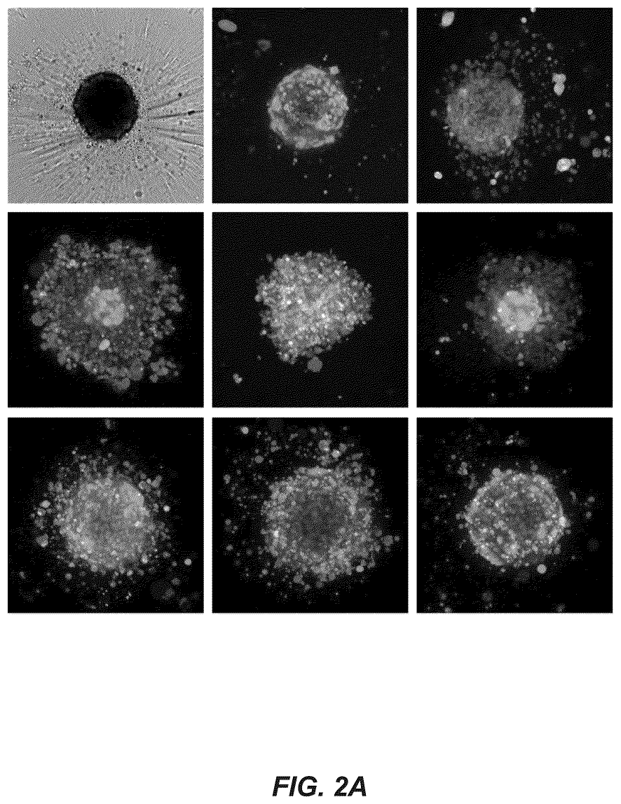

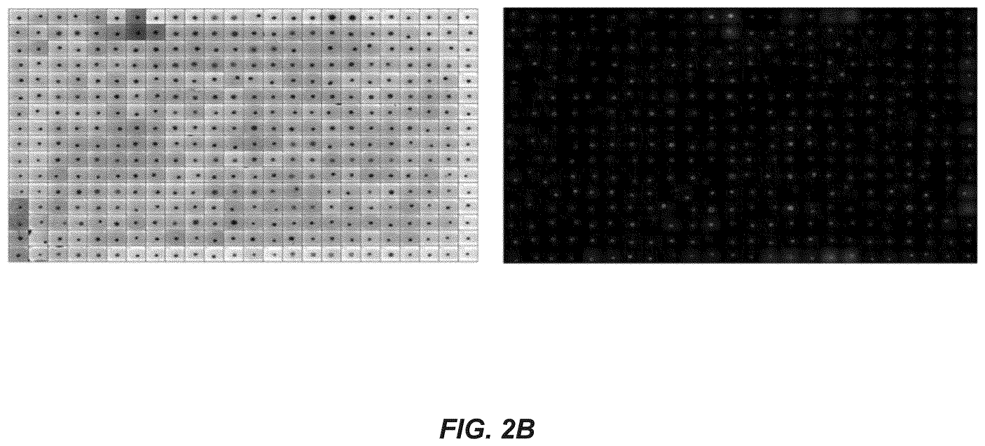

In the present disclosure, method development was focused on drug screening using primary-derived tumor cells in 3D culture. The goal of this study was to develop and evaluate fast, accurate, and reproducible high-content imaging methods to investigate effects of anti-cancer compounds on the morphology and viability of 3D cultures using live and fixed cells. In this study, the workflow was evaluated and optimized while characterizing several endpoint assays to test for general and mechanism-specific cytotoxicity of anti-cancer drugs (FIG. 1). Low-attachment, U-shaped, black-walled, clear-bottom 384-well plates may be used to simplify cell culture, compound addition, and imaging. Sirenko et al., Assay Drug Dev Technol. 2016; 14(7):381-94. Cells aggregate at the bottom of the U-shaped wells and form tumoroids centered in the well within about 48 hours. The thin plastic bottom of each well aids focusing and image acquisition with standard automated imaging systems. Entire tumoroids can be captured in one 10× or 20× image. In preliminary tests, reproducibility of spheroid formation and dependence of the spheroid size on the number of plated cells was studied. Cells were plated at different densities (500-8,000 cells/well), incubated for 48 h, and then imaged using TL imaging. It was found by visual assessment that a plating density of 2,000-3000 cells/well resulted in consistent tumoroid size and shape, with sizes suitable for image acquisition and analysis (diameter ˜200-300 mm). A plating density of 2000 cells/well was used for subsequent assays. At this density, the average tumoroid maximum diameter after 2 days was consistent as measured by transmitted light (TL) imaging, with a value of 270+/−37 mm (n=96) yielding a coefficient of variation of 13% (FIG. 2).

Tumoroids were then treated with 168 compounds from the NCI (National Cancer Institute) library of approved anti-cancer drugs. Five concentrations were used for testing: 10 nM, 100 nM, 1 mM, 10 mM and 100 mM. During screening, each compound was tested in duplicate, with one concentration per plate. In addition, positive and negative controls were included in the test plates. Positive controls included romidepsin, a compound that has shown high efficacy in previous tests. Cromwell et al., SLAS technology, 26(3), 237-248.

In the present disclosure, negative drug controls included acetaminophen, as well as multiple replicates of DMSO and media controls.

Automation using a liquid handler was used for compound dilutions, cell treatments, and staining. The schematic diagram of the process is shown in FIG. 1.

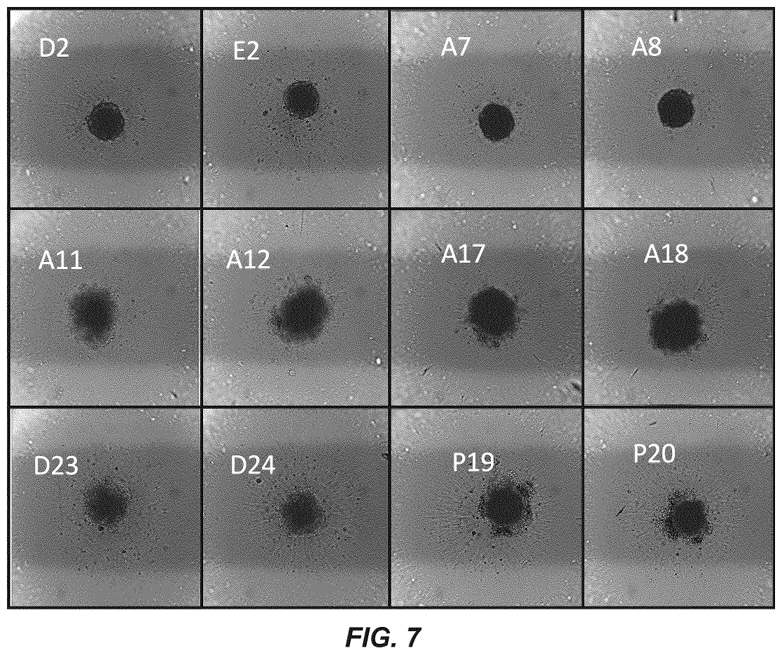

During incubation with compounds, tumoroids may be monitored daily using TL imaging. Image analysis may allow characterization of tumoroid size, diameter, compactness, and integrity, as well as optical density (FIG. 2B, FIG. 7). Images of tumoroids may be taken prior to compound treatment, and during treatment, e.g., day 3 and day 5 of compound treatment. For the end point assay, tumoroids may be stained with viability and nuclear dyes, e.g., on day 5 of compound treatment. Cells may be stained with a combination of Hoechst, calcein AM, and Ethidium Homodimer III (EthD-III) and analyzed using high content imaging for complex phenotypic analysis; a subset of plates was set aside for additional assays including Cell Painting.

### Methods and Results

Cell models. In the present disclosure a compound screening method was developed that uses primary tissue-derived tumoroids. However, any appropriate 3D target cell model may be employed. The cell model may be derived from a patient tissue, tumor, biopsy sample, or a tumoroid fragment. The 3D target cell model may be selected from spheroid, tumoroid, organoid, and PDX-derived organoids. The cell model may be provided comprising obtaining isolated cells from a primary tumor of a patient; cultivating the isolated cells to obtain two-dimensional (2D) cultivated cancer cells or passaging into murine models for expansion to provide xenograft cancer cells; and forming the 3D target cell model from the 2D or xenograft cancer cells.

As described in the present examples, tumoroids were formed from TU-BcX-4IC cell line derived from primary tumor samples as previously described. Briefly, patient-derived tumor samples were implanted into SCID mice, serially passaged, and then expanded in 2D culture. Matossian et al., 2021, Clinical & translational oncology, 10.1007/s12094-021-02677-8.

TU-BcX-4IC represents metaplastic breast cancer with a triple-negative breast cancer subtype and is an example of a highly heterogeneous phenotype of breast cancer. TNBC tumors have an aggressive clinical presentation due to high rates of metastasis, recurrence and chemoresistance. The original patient's tumor for the TU-BcX-4IC model exhibited rapid pre-operative growth despite conventional combination therapy with Adriamycin (doxorubicin), cyclophosphamide and paclitaxel. Tumoroids were formed from 2D TU-BcX-4IC cells as described in Methods.

Development and Optimization of the Live Cell High-Content Assay with 3D Spheroid Cultures.

As described in the present examples, the focus was on method development for drug screening using primary-derived tumor cells in 3D culture. The goal of this study was to develop and evaluate fast, accurate, and reproducible high-content imaging methods to investigate effects of anti-cancer compounds on the morphology and viability of 3D cultures using live and fixed cells. In this study, the workflow was evaluated and optimized while characterizing several endpoint assays to test for general and mechanism-specific cytotoxicity of anti-cancer drugs (FIG. 1). Low-attachment, U-shaped, black-walled, clear-bottom 384-well plates were employed to simplify cell culture, compound addition, and imaging. Sirenko et al., Assay Drug Dev Technol. 2016; 14(7):381-94.

Cells aggregate at the bottom of the U-shaped wells and form tumoroids centered in the well within 48 hours. The thin plastic bottom of each well aids focusing and image acquisition with standard automated imaging systems. Entire tumoroids can be captured in one 10× or 20× image. In preliminary tests, reproducibility of spheroid formation and dependence of the spheroid size on the number of plated cells was studied. Cells were plated at different densities (500-8,000 cells/well), incubated for 48 h, and then imaged using TL imaging. It was found by visual assessment that a plating density of 2,000-3000 cells/well resulted in consistent tumoroid size and shape, with sizes suitable for image acquisition and analysis (diameter ˜200-300 mm). A plating density of 2000 cells/well was used for subsequent assays. At this density, the average tumoroid maximum diameter after 2 days was consistent as measured by transmitted light (TL) imaging, with a value of 270+/−37 mm (n=96) yielding a coefficient of variation of 13% (FIG. 2).

Tumoroids may be treated with a multiplicity of candidate compounds. In the present examples, tumoroids were treated with 168 compounds from the NCI (National Cancer Institute) library of approved anti-cancer drugs. Five concentrations were used for testing: 10 nM, 100 nM, 1 mM, 10 mM and 100 mM. During screening, each compound was tested in duplicate, with one concentration per plate. In addition, positive and negative controls were included in the test plates. Positive controls included romidepsin, a compound that has shown high efficacy in previous tests. Cromwell et al., 2021, SLAS technology, 26(3), 237-248.

Negative controls included acetaminophen, as well as multiple replicates of DMSO and media controls. Automation using a liquid handler was used for compound dilutions, cell treatments, and staining. The schematic diagram of the process is shown in FIG. 1.

During incubation with compounds, tumoroids were monitored daily using TL imaging. Image analysis allowed characterization of tumoroid size, diameter, compactness, and integrity, as well as optical density (FIG. 2B, FIG. 7). Images of tumoroids were taken prior to compound treatment, and during day 3 and day 5 of compound treatment. For the end point assay, tumoroids were stained with viability and nuclear dyes on day 5 of compound treatment. Cells were stained with a combination of Hoechst, calcein AM, and Ethidium Homodimer III (EthD-III) and analyzed using high content imaging for complex phenotypic analysis; a subset of plates was set aside for additional assays including Cell Painting.

### High Content Imaging and Automation of the Screening Workflow.

The main method used for evaluation of phenotypic changes may be confocal fluorescent imaging of tumoroids using an automated imaging system. As described in the present examples, the ImageXpress confocal imaging system (FIG. 2A). The method was previously described in Sirenko 2015, Assay Drug Dev Technol. 2015; 13(7): 402-414. Images of organoids were taken with a 10× objective using Z-stack with a step size of 8 mm. Nuclei, live cells, and dead cells of stained cells were detected using DAPI, FITC, and TexasRed channels respectively. Maximum projection images were created from Z-stacks and analyzed as described in Methods. Multiple read-outs were generated characterizing spheroid area, diameter, intensities, and cell viability. In addition, cells inside each spheroid were counted using a nuclear stain. This last output was found to produce the best results. 3D culture, treatment, and end point assays are notably more complex than traditional 2D culture assays. Automated imaging and analysis of organoids are important for quantitative assessment of phenotypic changes in organoids, and for increasing throughput for experiments and tests. An automated, integrated system was built that allows monitoring, maintenance, and characterization of growth and differentiation of organoids and stem cells, as well as testing the effects of various compounds (FIG. 1). Suspended cells were plated into U-shape 384 well plates using a Biomek i7 automated liquid handler, incubated for 48 hours, and then treated with compounds, stained, and imaged. The protocols for compound additions, media, and staining included gentle media dispensing processes. Consecutively, tumoroids were cultured and monitored by automated imaging. Additionally, use of a collaborative robot enabled moving plates from the incubator to the imaging system, where transmitted light imaging or fluorescent imaging protocols were initiated.

### Observations of Phenotypic Changes and Criteria for Hit Selection.

The phenotypic changes for selected compounds may be derived from imaging analysis. Images obtained as described in the present examples are shown in FIG. 2. The phenotypic readouts included cell viability assessment and characterization of organoid and cell areas, and total cell count, along with characterization of nuclear area and fluorescence intensity. Advantages of this method included simplicity, reduced cost, and ease of workflow. In contrast to previous 3D spheroid studies performed using cell lines as described in Sirenko 2015, Assay Drug Dev Technol. 2015; 13(7): 402-414, 3D tumoroids formed from primary cells (TU-BcX-4IC cell line) did not increase in size during culture. However, upon drug treatment it was observed concentration-dependent disintegration of tumoroids (FIG. 2), as well as a decrease in the number of viable, Calcein AM positive cells and an increase in the number of dead, EthD-III positive cells.

Multiple quantitative descriptors may be characterized that can be used for studying tumor phenotypes and compound effects, including characterization of size and integrity, cell morphology and viability, as well as determining the presence of various cell markers. In some cases, the phenotype of organoid disintegration may be the most prominent. In the present examples, organoid disintegration was observed at lower concentrations than the increase in the number of dead cells, so that read-out was used to characterize concentration dependencies and compare effective concentrations for different compounds.

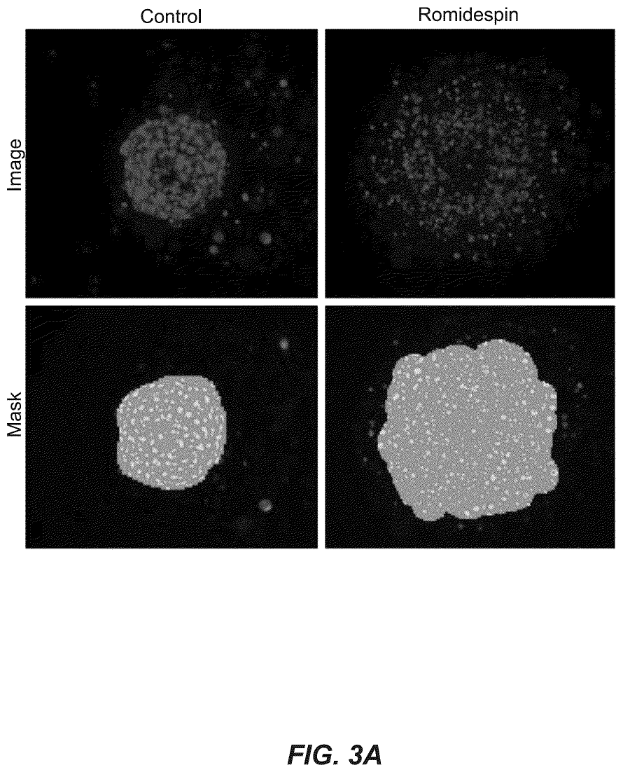

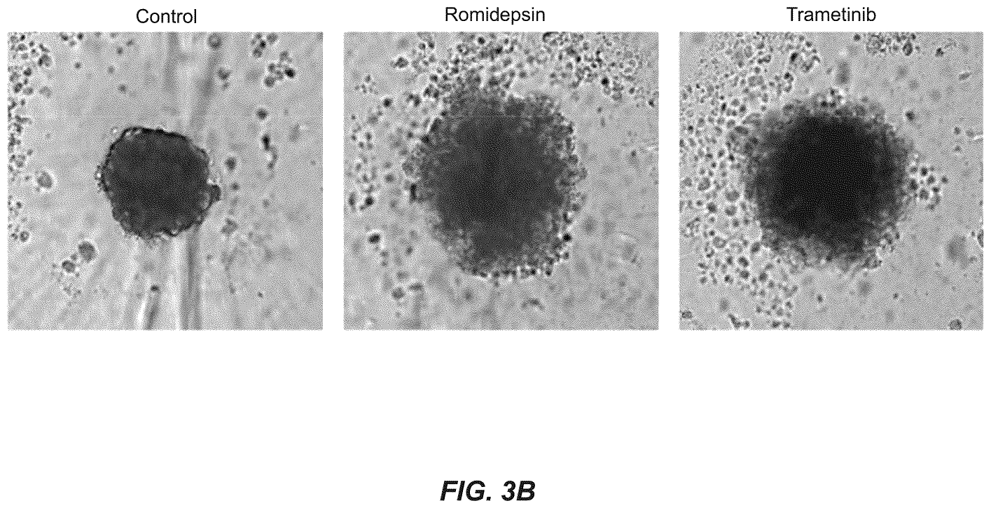

For screening the compound library, maximum projection images may be employed. For example, as shown in FIG. 3. Spheroid disintegration is visible as an increase of spheroid size, in contrast to nicely shaped, round images of intact spheroids. Tumoroid disintegration can be described by several measurements: for example, increase in organoid area/diameter. Most accurate measurements may be done by counting cells or nuclei inside organoids using maximum projection images. As seen in FIG. 3, compact tumoroids had a smaller projected area and also a lower nuclear count in the projection images (yellow dots). Disintegrated tumoroids are made up of cells which are not held together in a tight aggregate but instead are loosely piled up in the well bottom resulting in 1) an increased projected area and 2) an increase in nuclear count in the projection images. It is important to note that the apparent increase in cell number represents a feature of analysis using 2D projection images and the fact that tumoroids are falling apart and the individual cells are not tightly associated with each other.

While counting cells in 3D instead of using 2D projection may better reflect the true cell count (data not shown), that may not allow quantitation of organoid disintegration. This method may be better suited for screening experiments because of the reduced demand for data storage and reduction in time for 3D analysis. Simple nuclei count analysis in projection images was used as a reliable surrogate marker for organoid integrity/dis-integration. Therefore, in the analysis of projection images, the increase of nuclear count was used as a read-out for tumoroid dis-integration (Table 5). Notably, selected drugs (e.g., doxorubicin and several others) resulted in nuclei damage, and as a result, a decrease in counted nuclei. The AVERAGE+/−2STDEV from the DMSO samples was used to flag affected wells. The hits were confirmed through referencing the primary images to exclude experimental artifacts (e.g., focus failure, pipetting errors, rolling the object off center, etc.) which accounted for approximately 1% of wells.

Results from Library Screening and Secondary Screen.

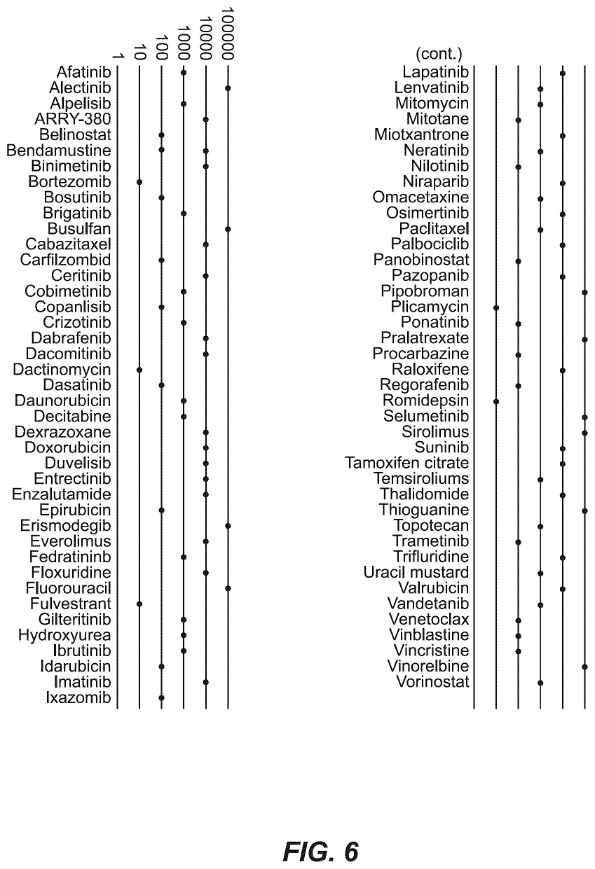

The effects of 168 compounds from the NCI library were tested with 5 concentrations listed above and described in the examples. Then assessment of tumoroid integrity by nuclear count was used to identify hits across different concentrations. Several drugs were identified that demonstrated efficacy of targeting tumor subtypes resistant to traditional cancer therapy. Drugs listed in Table 1 demonstrated efficacy by affecting tumoroid phenotypes at indicated concentrations (same concentrations had efficacy at higher concentrations). Interestingly, several compounds were found to have efficacy at low concentrations, which may be valuable for the development of potential therapeutics. Those include romidepsin, dactinomycin, plicamycin, bortezomib, and dasatinib. A total of 33 compounds were selected as “hits” for concentrations 10-1000 nM, while an additional 29 compounds had significant effects at 10 mM. (FIG. 6). Because greater than 300 of compounds had some effects at a concentration of 100 mM, this data point was considered less informative.

TABLE 1
Compounds effective in screening assay at low concentrations.
Compounds were selected as described in Results section.

| | Effective | | |
|---|---|---|---|
| Compound | concentration | | Mechanism of action |
| Dactinomycin | 10 | nM | Intercalation of DNA |
| Plicamycin | 10 | nM | DNA/RNA polymerase inhibitor, |
| | | | hepatotoxicity led to discontinue in |
| | | | 2000 |
| Romidepsin | 10 | nM | HDAC 1/2 inhibitor |
| Bortezomib | 10 | nM | Inhibits the catalytic site of the 26S |
| | | | proteasome |
| Fulvestrant | 10 | nM | Selective Estrogen receptor |
| | | | degrader |
| Dasatinib | 100 | nM | Tyrosine Kinase Inhibitor for |
| | | | BCR/Abl fusion |
| Mitotane | 100 | nM | Steroidogenesis inhibitor used for |
| | | | adrenal carcinoma and Cushing's |
| Vinblastine sulfate | 100 | nM | Binds tubulin, disrupts |
| | | | microtubules |
| Vincristine sulfate | 100 | nM | Binds tubulin, disrupts |
| | | | microtubules |
| Carfilzomib | 100 | nM | Inhibits the 20s proteasome |
| Trametinib | 100 | nM | Inhibits MEK1 and MEK2 |
| Ixazomib citrate | 100 | nM | Inhibits proteasome subunit beta 5, |
| | | | same mechanism as bortezomib, |
| | | | related to carfilzomib |
| Ponatinib | 100 | nM | Inhibitor for BCR/Abl fusion, |
| | | | second line after dasatinib or |
| | | | imatinib |
| Belinostat | 100 | nM | Pan- HDAC inhibitor |
| Panobinostat | 100 | nM | Pan- HDAC inhibitor |
| Regorafenib | 100 | nM | Tyrosine Kinase Inhibitor of |
| | | | VEGFR-TIE2 |
| Bosutinib | 100 | nM | Tyrosine Kinase Inhibitor of Abl |
| | | | and Src kinases |
| Copanlisib tris-HCl | 100 | nM | PI3K inhibitor |
| Mitomycin | 1 | uM | Alkylating agent- DNA |
| | | | crosslinking |
| Uracil mustard | 1 | uM | Alkylating agent- DNA |
| | | | crosslinking |
| Omacetaxine | 1 | uM | Inhibits translation (blocks tRNAs) |
| Daunorubicin | 1 | uM | Intercalation of DNA |
| hydrochloride | | | |
| Vandetanib | 1 | uM | Inhibits VEGFR, EGFR, RET |
| Paclitaxel | 1 | uM | Stabilizes microtubes, inhibits |
| | | | spindle formation |
| Ibrutinib | 1 | uM | Inhibits Bruton's tyrosine kinase |
| Idarubicin | 1 | uM | Daunorubicin analog |
| hydrochloride | | | |
| Venetoclax | 1 | uM | Inhibits BCL2 |
| Epirubicin | 1 | uM | Intercalation of DNA |
| hydrochloride | | | |
| Fedratinib | 1 | uM | JAK2 selective inhibitor |
| Temsirolimus | 1 | uM | Inhibits mTOR |
| Vorinostat | 1 | uM | Inhibits HDAC1, HDAC2, |
| | | | HDAC3, HDAC6 |
| Brigatinib | 1 | uM | Inhibits ALK, EGFR |
| Gilteritinib | 1 | uM | Inhibits FLT3 receptor |

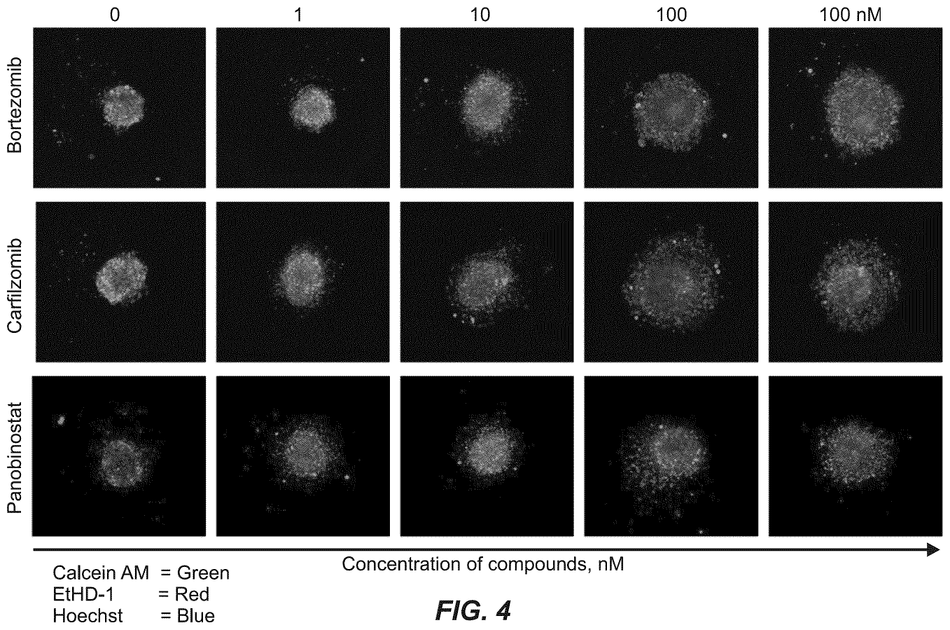

To confirm the findings in the screening assay, a subset of compounds was selected for secondary follow-up analysis. 10 compounds, including panobinostat, carfilzomib, and bortezomib were tested across 7 concentrations in the 1-10000 nM range. Spheroids were treated for 5 days with these compounds, then stained as described above (FIG. 4). Dose-dependent cell death and disintegration of microtissues was observed. EC₅₀ values (Table 2) were determined using tumoroid disintegration measurement (nuclear count in projection images). All tested compounds showed similar activities as observed in the initial compound screen.

TABLE 2
EC₅₀ values of indicated compounds in 3D assay
determined by 7-point concentration curves.

| Compound | EC₅₀, nM |
|---|---|
| Bortezomib | 17 |
| Carfilzomib | 16 |
| Copanlisib | 28 |
| Romidepsin | 36 |
| Panobinostat | 54 |
| Trametinib | 70 |
| Omacetaxine | <100 |
| Carfilzomib | <100 |
| Crizotinib | >1000 |
| Sunitinib | 1700 |
| Taxol | 2900 |
| Celecoxib | no effect |

### Monitoring Phenotypic Changes Using Transmitted Light.

Notably, changes in tumoroid phenotypes upon compound treatments were also observed with TL imaging, and artificial intelligence (AI) tools were used to identify and classify different tumoroid phenotypes. A deep learning-based segmentation (IN Carta Image Analysis Software) approach was used to find all tumoroid objects in transmitted light, either intact or affected. Following segmentation and data extraction, a machine learning tool was utilized to classify all tumoroids into intact, intermediate or severely affected categories. Affected tumoroids were flagged by increase in the object areas (criteria were set as AVERAGE 2*STDEV from the DMSO-treated), allowing detection of effective compounds (FIG. 7, Table 6). An advantage of this method is that compound effects could be interrogated in a label-free manner during multiple time points. However, the analysis allowed some ambiguity in determining accurate areas of the objects, especially when the phenotypic changes were weak or moderate (FIG. 7). The endpoint fluorescent imaging and analysis enabled more robust measurements and compound selection (Tables 5 and 6).

### Evaluation of Phenotypic Changes by Cell Painting Assay.

For a more in-depth investigation into cytotoxic mechanisms elicited by the compounds assayed, other analysis methods were performed in parallel to fully characterize phenotypic changes detected. The Cell Painting assay was adapted for 3D tumoroids for the evaluation of compound effects on tumoroid phenotype. The method uses up to six or more fluorescent dyes to label different cellular components or organelles: e.g., nuclei, nucleoli, RNA, endoplasmic reticulum, mitochondria, plasma membrane, Golgi and cytoskeleton. (Rohban et al., 2017, eLife, 6, e24060; Ljosa et al., Journal of biomolecular screening, 18(10), 1321-1329; Gustafsdottir et al., 2013, PloS one, 8(12), e80999; Bray et al., 2016, Nature protocols, 11(9), 1757-1774).

This method, which was developed for use in 2D assay systems has been successfully used for phenotypic profiling to provide insights into functional genomics applications and mechanism of action of novel compounds, and to reveal subtle effects of various drugs and small molecules on cell health. Here, the Cell Painting assay was adapted and further developed for 3D cell culture models.

For phenotypic profiling, the tumoroids were treated with a single 10 μM concentration of compound, using the methods described above. Notable changes in the staining protocol included increased times for dye incubation, fixation, and permeabilization. A challenge of the assay is to minimize the crosstalk of multiple dyes, which we achieved by using lasers as the illumination light source, with a narrow spectrum for each wavelength and minimum overlap between stains. In the original Cell Painting assay, the Golgi and actin filament images were acquired in the same imaging channel. This created additional hurdles, as it was challenging to extract measurements from the Golgi compartment separately from the actin structures. To improve the resolution between the Golgi and actin compartments, Alexa Fluor 568 was swapped out for Alexa Fluor 750 phalloidin, which allows for the cytoskeleton to be imaged in a different channel from the Golgi compartment by using the far-red laser on our imager. Images were acquired with Z-stacks using 20× magnification, and the analysis was carried out on projection (maximum) images.

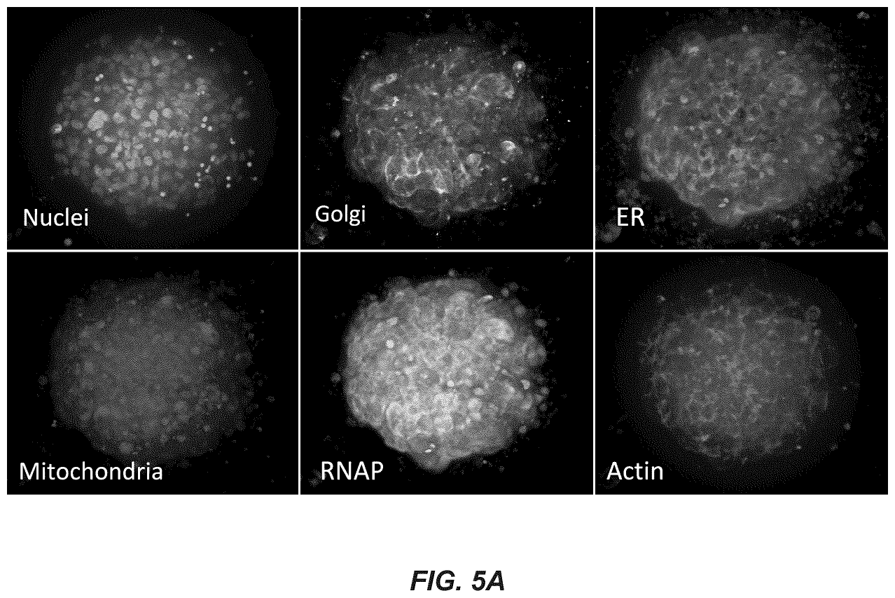

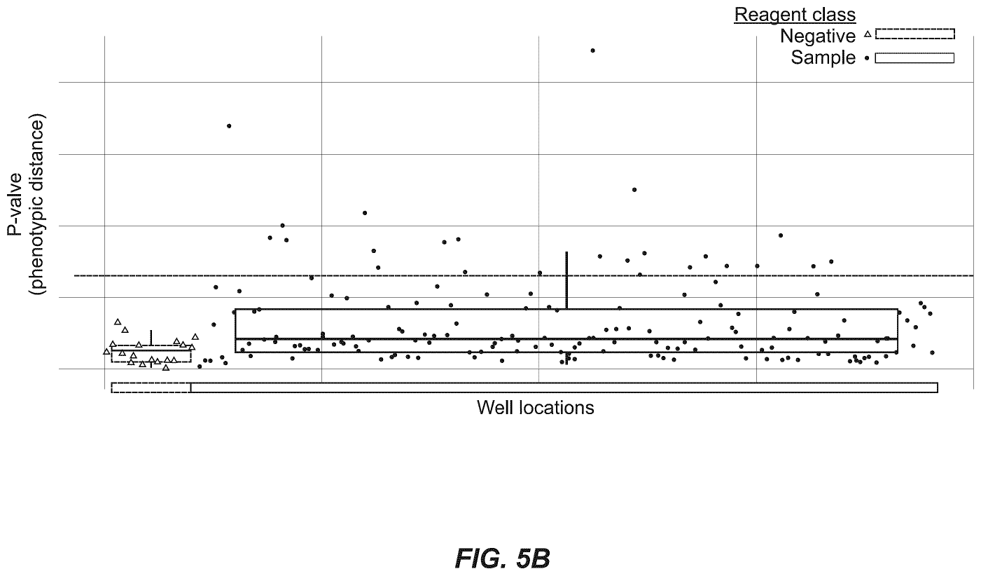

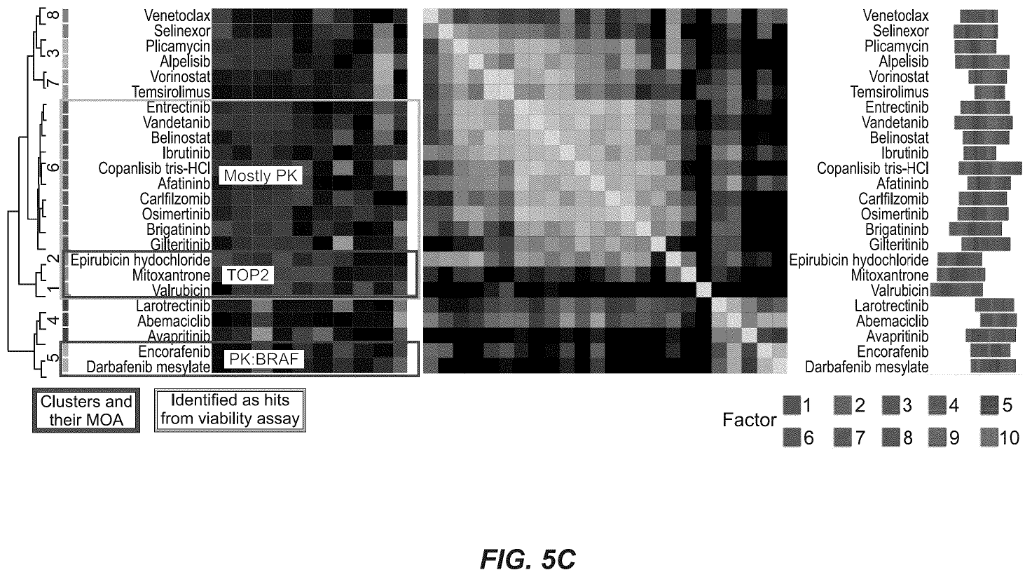

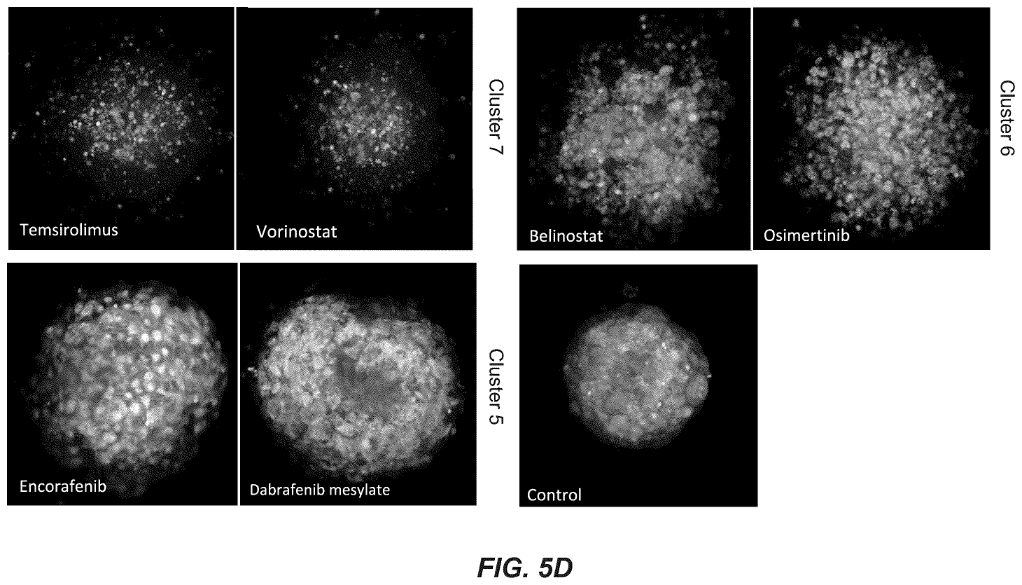

Unlike the previous analysis that included cell count or live-dead evaluation, multiple read-outs may be collected from the whole spheroid to form their phenotypic profile. Because the added compounds may affect staining patterns, it may be difficult to achieve good spheroid segmentation based solely on the fluorescent images. Instead, the spheroid images may also acquired in transmitted light (bright field), and these images may be used to identify the spheroid structures using a deep learning-based image segmentation approach which may improve the analysis. Features extracted from each spheroid may include object morphologies, intensities, and texture measurements for, e.g., the six cell stains and bright field images. 202 measurements per spheroid were uploaded into StratoMineR software (CoreLife Analytics) for data analysis to identify hits and for cluster analysis based on similarity of their phenotypic profiles. FIG. 5 presents a dendrogram showing clustering of tested compounds based on phenotype similarity of the spheroids.

To determine hits from the assay, a phenotypic distance score may be calculated based on the PCA (principal component analysis) components. (Perlman et al., Multidimensional drug profiling by automated microscopy. Science (New York, N.Y.), 306(5699), 1194-1198; Omta et al., 2016, Assay and drug development technologies, 14(8), 439-452).

This score is a measure of the phenotypic effect of the compounds on the tumoroids relative to the controls. All hits may then be clustered based on their phenotypic profiles using the normalized principal component scores. In the present examples, 24 hits (Table 3) were identified (p<0.05) as being significantly different from the DMSO control tumoroids (FIG. 5). A reasonable correlation was obtained between the viability assay and the Cell Painting assay.

From the Cell Painting assay, 67% (16), or two-thirds of the hits were also identified in the viability/spheroid disintegration assay. Hits from the viability assay were found mostly in the same clusters (clusters 1, 2 and 6, FIG. 5), suggesting that these spheroids display phenotypic profiles that are associated with most notable cytotoxicity. For example, all compounds in clusters 1 and 2 are hits also flagged in the viability assay, and they are known to affect topoisomerase 2. The other hits from the Cell Painting assay included compounds with less obvious cytotoxic effects, but these hits were still phenotypically different from the controls and interestingly, many of these compounds are known to affect various protein kinase pathways. These results are consistent with the fact that while cytoxicity changes phenotypes very dramatically, more subtle changes can be detected using phenotypic profiling.

TABLE 3
Compounds effective in the Cell Painting assay. Compounds
selected as described in the Results section.

| Compound | Distance | | Cluster | Viability |
|---|---|---|---|---|
| (10 μM) | Score | p-value | ID | HIT? |
| Dabrafenib | 6.248831502 | 0.014165 | 5 | yes |
| mesylate | | | | |
| Encorafenib | 5.516036128 | 0.015028 | 5 | no |
| Avapritinib | 5.386292153 | 0.015154 | 6 | no |
| Abemaciclib | 5.819034729 | 0.006458 | 4 | no |
| Larotrectinib | 5.666943776 | 0.0095 | 4 | no |
| Valrubicin | 8.136000201 | 3.22E−05 | 1 | yes |
| Mitoxantrone | 6.687853572 | 0.000375 | 2 | yes |
| Epirubicin | 5.883205304 | 0.012997 | 2 | yes |
| hydrochloride | | | | |
| Afatinib | 5.046845431 | 0.026011 | 7 | yes |
| Copanlisib tris- | 5.146853912 | 0.037712 | 7 | yes |
| HCl | | | | |
| Ibrutinib | 4.93823624 | 0.04635 | 7 | yes |
| Belinostat | 5.348047631 | 0.017714 | 7 | yes |
| Vandetanib | 5.184617965 | 0.0292 | 7 | yes |
| Entrectinib | 4.84271406 | 0.046947 | 7 | yes |
| Gilteritinib | 5.23882208 | 0.045371 | 7 | yes |
| Brigatinib | 5.771498629 | 0.021974 | 7 | yes |
| Osimertinib | 5.241373452 | 0.024226 | 7 | yes |
| Carfilzomib | 5.006962745 | 0.036124 | 7 | no |
| Temsirolimus | 5.699077848 | 0.026386 | 8 | no |
| Vorinostat | 5.078731963 | 0.036114 | 8 | no |
| Alpelisib | 5.833763084 | 0.003154 | 3 | yes |
| Plicamycin | 5.134541481 | 0.034984 | 3 | yes |
| Selinexor | 5.004358366 | 0.031059 | 9 | no |
| Venetoclax | 4.921513854 | 0.034861 | 9 | yes |

### Evaluation of Cell Viability Using ATP Read-Outs.

Cell metabolism may be measured by ATP level as another readout for effects of anti-cancer drugs that is related to cytotoxicity. In the present examples, a subset of 12 compounds were used for evaluation of compound effects, using the CellTiter-Glo 3D assay (Promega), which detects ATP levels. Compounds may be tested using 7 point 5× dilutions starting from 100 uM. In the present examples, compound effects on 2D culture and 3D cultures were compared, using imaging and CellTiter-Glo 3D methods. Cultures were set up in parallel, using same cell number (2000). Cultures were allowed to grow for 48 hours, then treated with compounds for 5 days. EC₅₀ values were calculated from concentration dependencies are presented in the Table 4. Data shows that there is significant consistency between EC₅₀ values obtained by imaging and CellTiter-Glo 3D readouts, while EC₅₀ values between 2D and 3D cultures varied for a number of compounds.

TABLE 4
Comparison of EC50 values obtained from 2D
and 3D assays by imaging or ATP assays.

| | EC50, mM | | | |
|---|---|---|---|---|
| Drug | ATP 3D | Imaging 3D | ATP 2D | Imaging 2D |
| Plicamycin | .018 +/− .02 | 0.156 | <.01 | <.01 |
| Dactinomycin | 0.005 | 0.033 | <0.01 | <.01 |
| Idarubicin | .145 +/− 0.012 | 3.93 | .067 +/− .009 | ~1 |
| Temsirolimus | no fit | 8.52 +/− 10.37 | no fit | 10 |
| Bortezomib | .015 +/− .001 | 0.07 | 0.01 | .01 +/− 0.002 |
| Trametinib | 2.62 +/− 1.214 | 8.82 | 7.33 +/− 4.31 | 6.27 +/− 3.55 |
| Panobinostat | .084 +/− .016 | 0.141 | .046 +/− .009 | .006 +/− .015 |
| Ibrutinib | 15.76 +/− 14.87 | ~20 | 25 +/− 2.4 | 20.2 |
| Carfilzomib | 5.82 +/− 2.24 | 0.073 | 1.04 +/− .10 | 1.05 +/− .31 |
| Romidepsin | ~.01 | <0.01 | ~.01 | ~.01 |

### Cell Metabolism Dynamics.

In the present examples, tumoroids treated with four compounds, paclitaxel, romidepsin, doxorubicin, and trametinib, were analyzed for lactate secretion. Elevation of lactate typically suggests a switch to aerobic glycolysis; tumor cells metabolize glucose into lactate even in the presence of high oxygen. (Yang et al., Sci Rep 2017; 7:43864).

Recent studies revealed that metabolic alterations of cancer cells play important roles in chemoresistance in breast cancer, and exposure of cancer cells to chemotherapeutics induces metabolic reprogramming toward increased glycolysis and lactate production. (Dong et al., Cell Commun Signal 2020; 18:167; Barnes et al., Br J Cancer 2020; 122:1298-308).

Therefore, monitoring lactate production over the course of treatment in conjunction with other response endpoints provides valuable information for understanding the dynamics of metabolic perturbations associated with drug response and resistance.

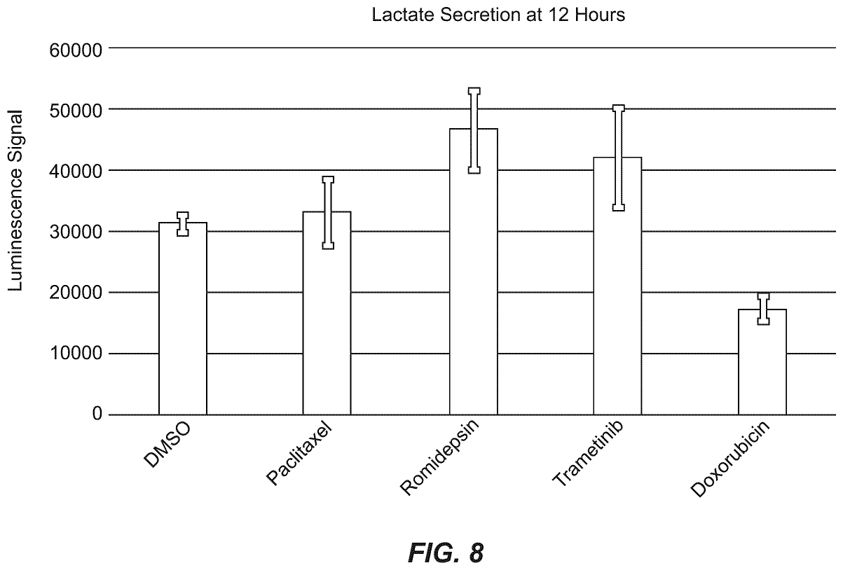

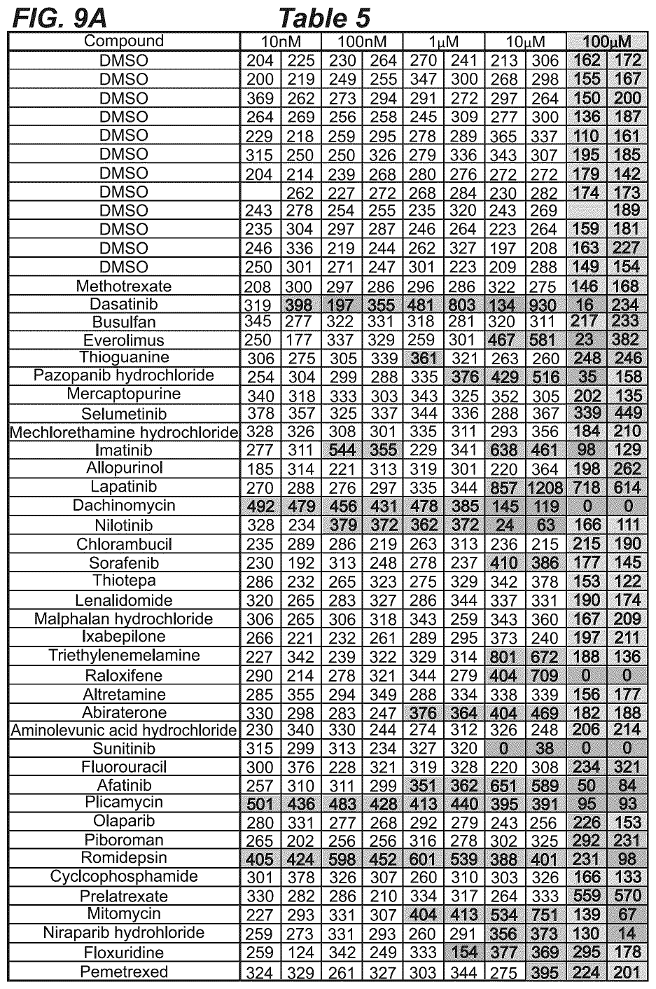

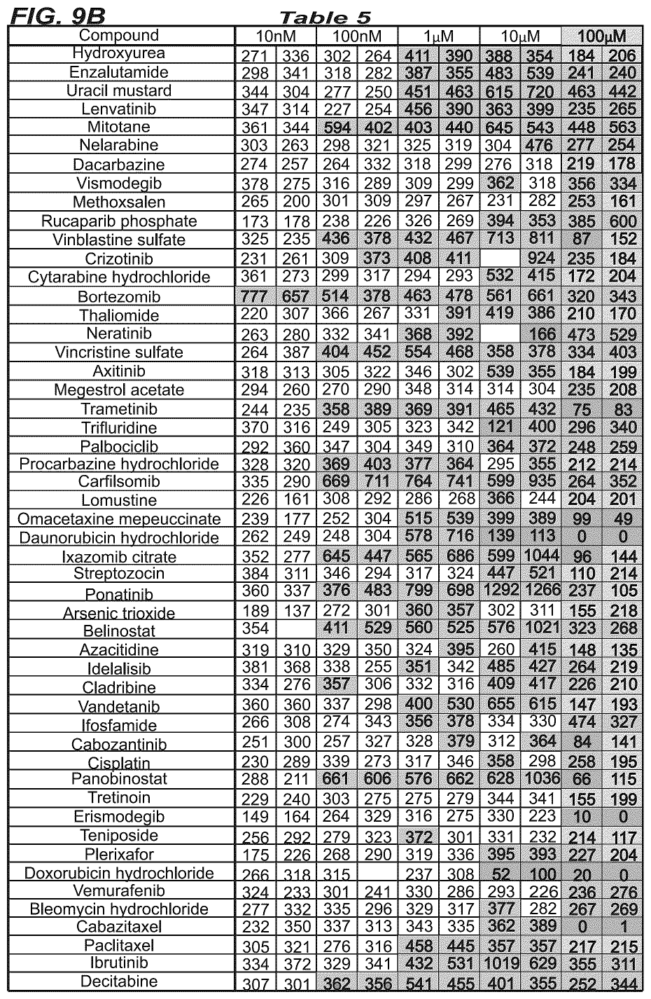

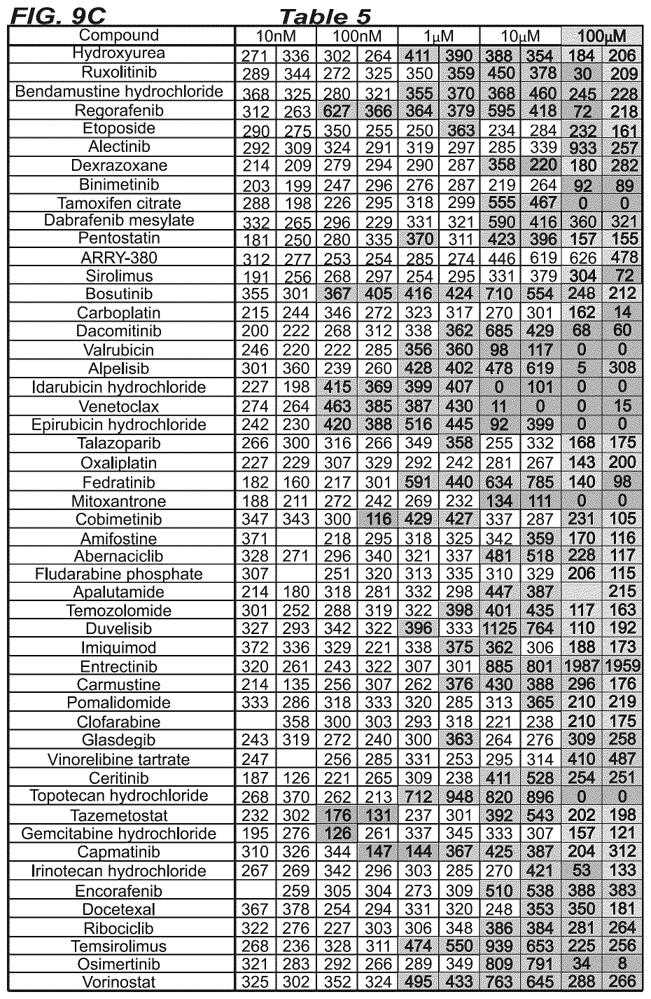

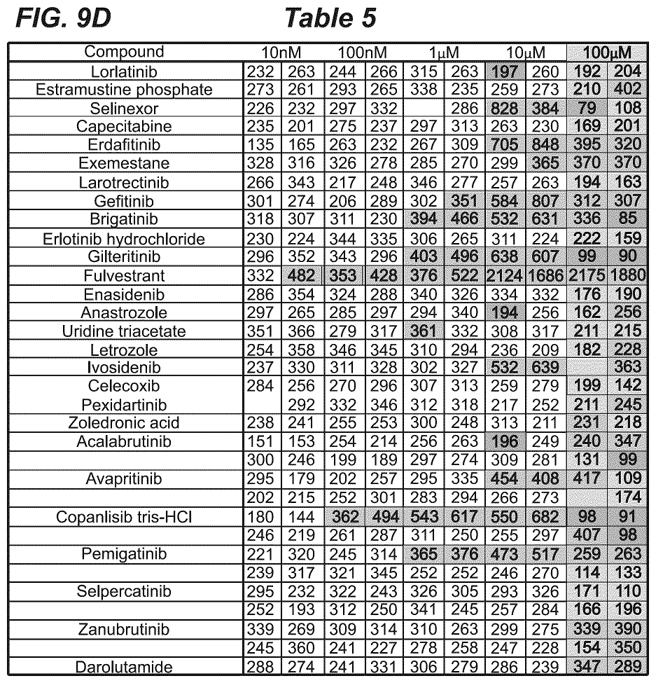

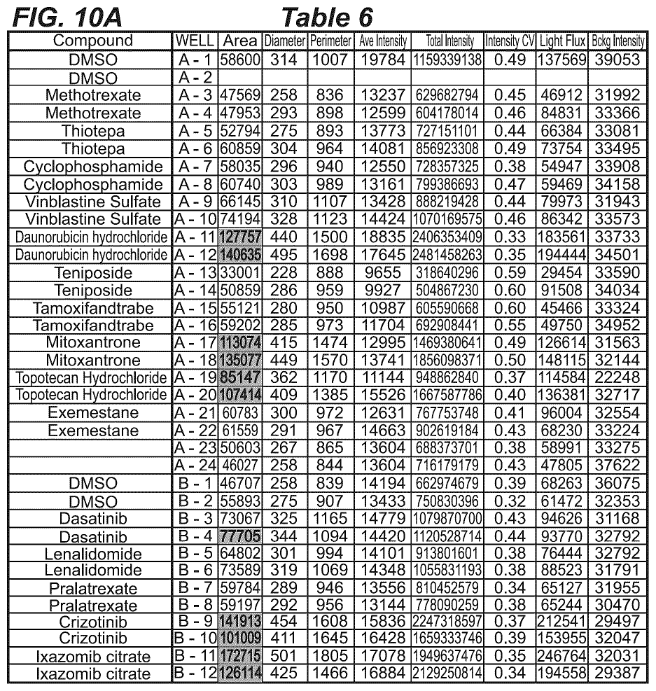

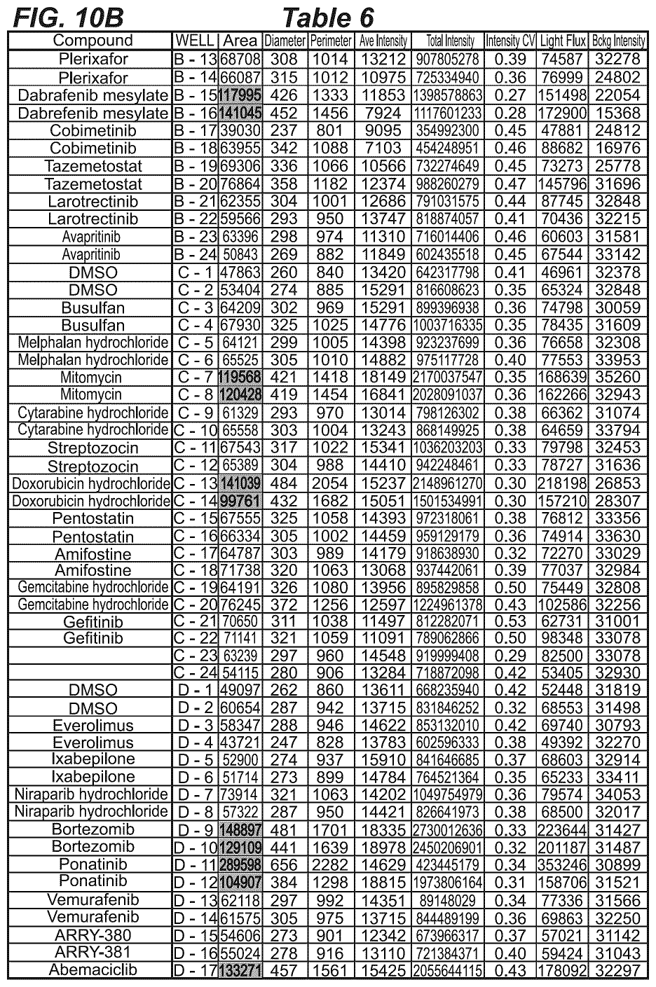

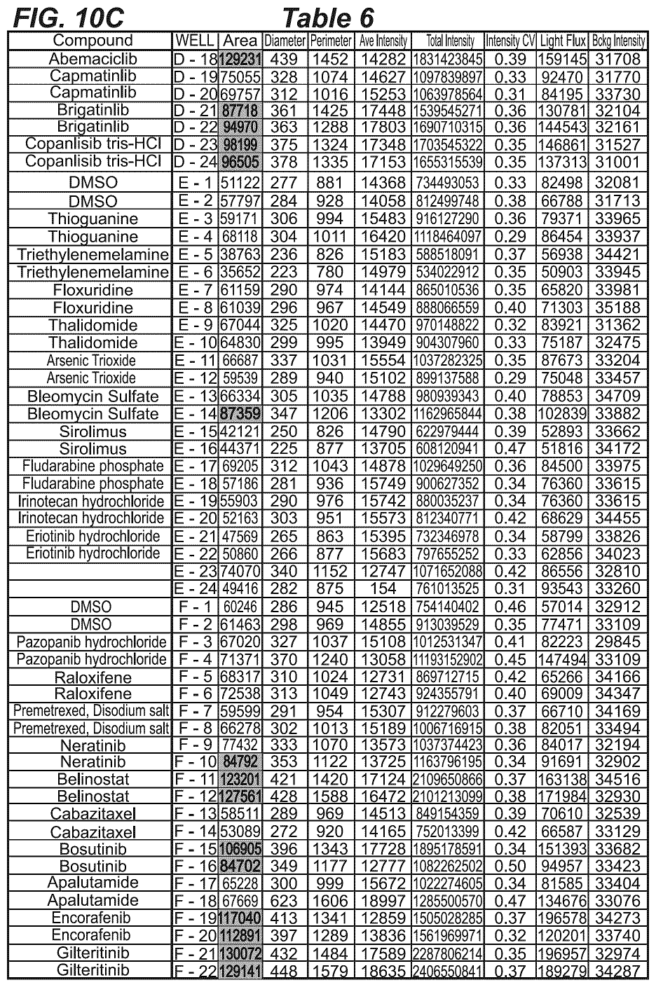

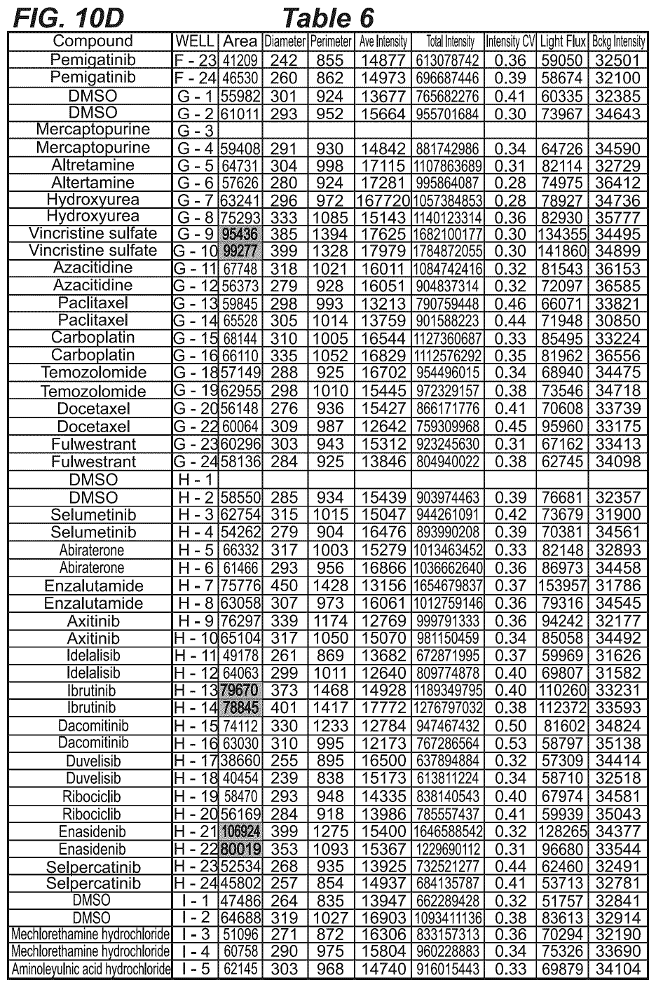

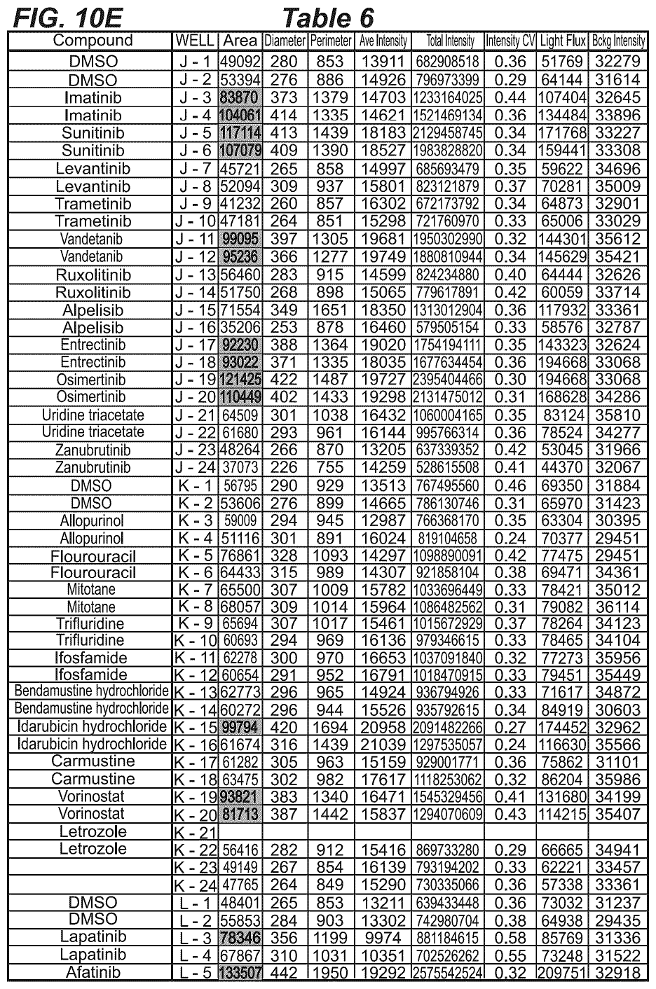

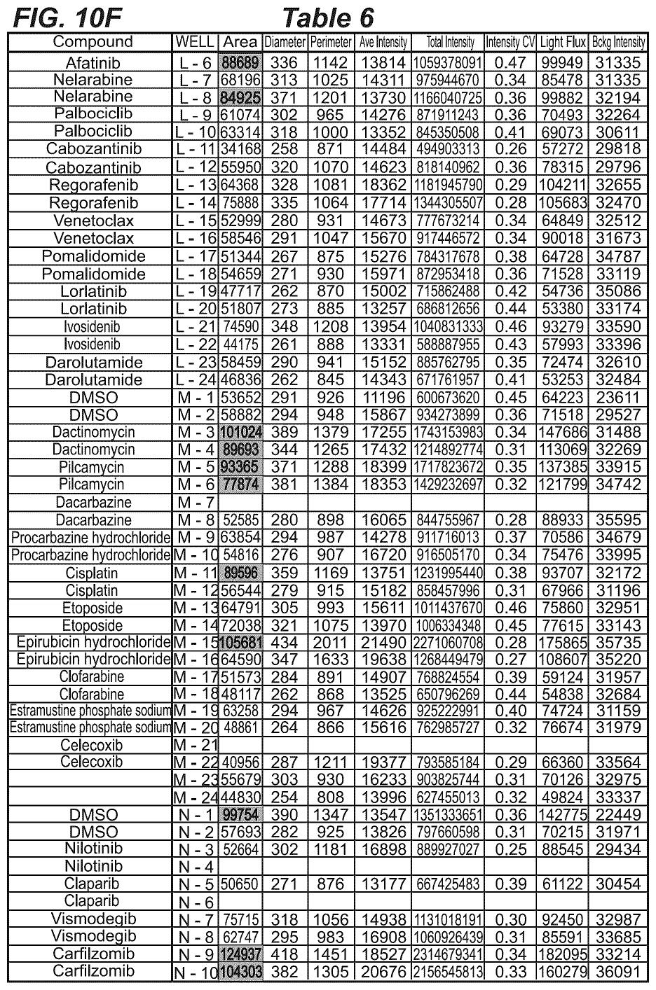

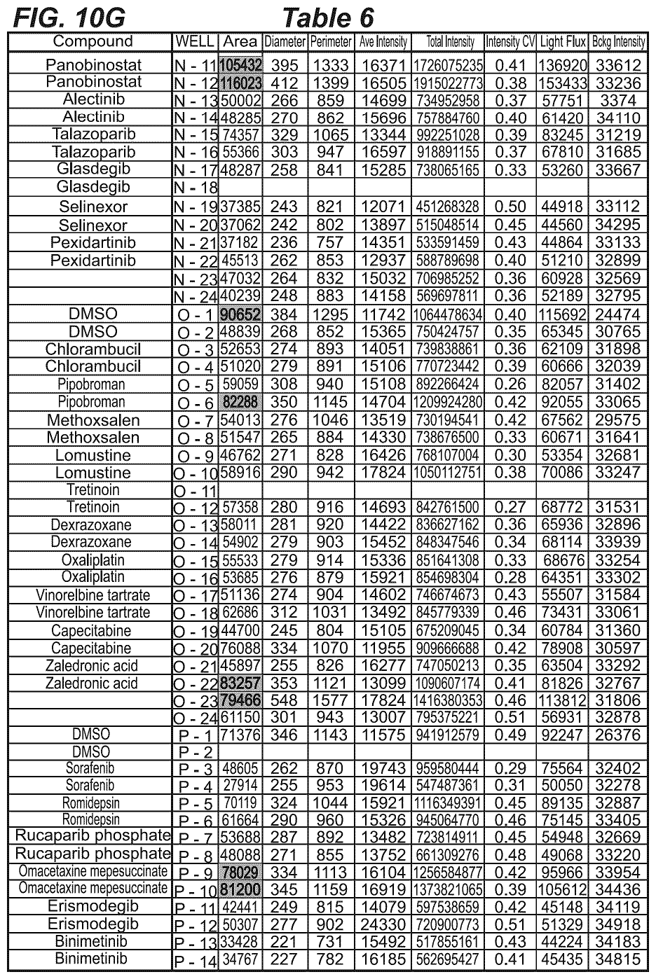

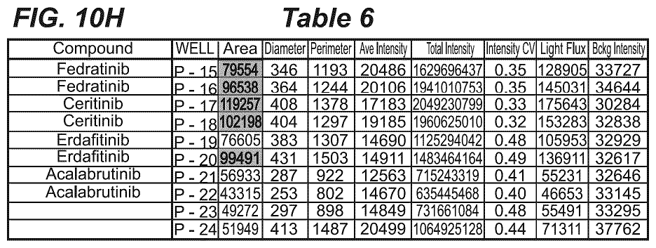

Compound treatments and secretion of lactate were studied using a Pu MA System, (Cromwell et al., 2022, SLAS Discovery 2022; 27(3): 191-200) a microfluidic based automated organoid assay platform that allows multistep protocols to be performed without disruption of or damage to tumoroids. Five supernatant samples were collected from treated tumoroids using flowchip automation over a 12-hour period. Two to three independent samples were collected, and supernatants were analyzed for lactate concentration using the Lactate-Glo assay (Promega). The lactate secretion results for the 12 hour time point are shown in FIG. 8. Two-way ANOVA revealed statistically significant increases in lactate secretion for romidepsin and trametinib. between baseline and measured time points for three out of the four compounds. The observations are in line with previously published reports showing increased lactate production for breast cancer cells in response to chemotherapeutic treatments. (Hou et al., Cancer Cell Int 2019; 19:193).

### EXAMPLES

### Example 1. Cell Model

Methods for generating tumoroids and PDX organoids (PDXO) were adapted from those previously described (Matossian et al., 2021). The primary tumor sample was implanted into SCID/Beige mice and exhibited rapid tumor growth, reaching maximal tumor volume (>1000 mm³) in 14 days. Then, a cell line generated from that sample was expanded in 2D culture (TU-BcX-4IC, also denoted as 4IC). Tumor spheroids, which are called tumoroids in this study, were formed from TU-BcX-4IC cells expanded in 2D. To form 3D tumoroids, TU-BcX-4IC cells were dispensed at ˜2,000 cells per well (in U-shape low attachment 384-well plates, Corning) and incubated for 48 hours until they formed tight tumoroids. 4IC cells were cultured with Advanced DMEM supplemented with glucose, non-essential amino acids (NEAA), 2 mM glutamine and insulin 120 μg/L, 10% FBS (Gibco 12491-015). For metabolic assays, tumoroids were cultured with DMEM+10% dialyzed serum (2 mM glutamine, 5 mM glucose, without phenol red).

Tumoroids were treated with compounds as follows. Compound libraries of approved anti-cancer drugs were obtained from NIH. Compound dilution plates were prepared using the Beckman liquid handling system. Five compound dilutions (20 nM, 200 nM, 2 uM, 20 uM and 200 uM) were prepared in 384 well plates. Then 50 ul of compound mixes were added to the tumoroid culture plates (50 uL) using a liquid handler programmed for slow dispensing of liquid. The final concentrations of compounds were 10 nM, 100 nM, 1 uM, 10 uM, and 100 uM. DMSO concentrations on each plate were matched for 0.05%, with the exception of 100 uM plates that had 0.5% DMSO. Each plate contained 32 DMSO only wells.

### Example 2. Drug Treatment

### Oncology Drug Set and Cell Treatments

TU-BcX-4IC cells were treated with the commercially available NCI Approved Oncology Drug set for 5 days total. On day 3, 50% of media was replaced with the fresh compound solutions. Cell cultures were treated with five concentrations, one concentration per plate, each compound in duplicates. Solution controls (DMSO) as well as a positive control (romidepsin) were included in each plate.

### Example 3. 3D Cell Monitoring and Imaging

Transmitted light (TL) and fluorescent (FL) images were acquired on the ImageXpress Confocal HT.ai High-Content Imaging System (Molecular Devices) and images analyzed using MetaXpress High-Content Image Analysis Software. Tumoroid images were acquired in TL with approximately 60 μm offset. Zstack images were acquired with the 10× objectives using confocal mode. Best focus projection images were used for TL analysis, and maximum projection images were used for FL analysis. MetaXpress Software or IN Carta Image Analysis Software was used for analysis.

### Example 4. Automation of 3D Cell Culture and Imaging

### Protocols

Automated imaging and analysis of organoids were employed for quantitative assessment of phenotypic changes in organoids, and for increasing throughput for experiments and tests. An automated, integrated system was built that allows for automated monitoring, maintenance, and characterization of growth and differentiation of organoids and stem cells, as well as testing the effects of various compounds. The automated system includes the ImageXpress Confocal HT.ai system and MetaXpress software (Molecular Devices), automated CO² incubator (LiCONiC), Biomek i7 Automated Workstation (Beckman Coulter Life Sciences), collaborative robot and rail. Robotic automation was enabled by Green Button Go Scheduler (BioSero).

### Example 5. Multiparametric Live Cell Toxicity Assay

The method for imaging and high content analysis of 3D spheroids was previously described (Cromwell et al., 2021., SLAS technology, 26(3), 237-248; Sirenko et al., 2016, Assay Drug Dev Technol. 2016; 14(7):381-94; Sirenko et al., 2015, Assay Drug Dev Technol. 2015; 13(7):402-414).

Briefly, following incubation with test compounds, spheroids were stained with a mixture of three dyes: 1 μM calcein AM, 3 μM EthD-III, and 33 μM Hoechst 33342 (Life Technologies, Carlsbad, CA). For selected experiments spheroids were also stained with FITC mouse anti-E-cadherin antibody (Cell Signaling Technology #3195, Danvers, MA) and PE anti-human CD44 antibody (BioLegend #338,808, San Diego, CA). Automated staining of spheroids was performed for one hour. After staining, dye solution was replaced with 1×PBS.

### Example 6. High-Content Imaging

Images were acquired using the ImageXpress Micro Confocal High-Content Imaging System (Molecular Devices, San Jose, CA), as previously described (Sirenko et al., 2016, Assay Drug Dev Technol. 2016; 14(7):381-94) with a 10× Plan Fluor or 20× Plan Apo objective. DAPI, FITC, and Texas Red filter sets were used for imaging. A stack of 7 to 15 images separated by 10 to 15 μm was acquired, starting at the well bottom and covering approximately the lower half of each spheroid. Typically, a Z-stack of images covered 100-200 μm for spheroids. Image analysis was performed either in 3D or using the 2D Projection (maximum projection) images of confocal image stacks. Transmitted light images were used for cell culture monitoring or protocol optimization.

To image the organoids labeled with the cell painting dyes, the ImageXpress Confocal HT.ai system, equipped with a laser light source, was used for image acquisition. The confocal mode (pinhole size 60 μm) was used to image organoids with a 20× Plan Apo Lambda, NA 0.75 objective, with z-stacking enabled (5p m step size). The maximum projection image was selected in the acquisition setup. Images were captured in six fluorescent channels and in transmitted light (bright field) in order of increasing fluorophore excitation wavelength (nm) to reduce crosstalk (DAPI 405/452, FITC 467.5/520, YFP 520/562, Texas Red 555/624, Cy5 638/692 and Cy7 725/794) (dye excitation nm/emission nm).

### Example 7. Organoid Image Analysis

Images were analyzed using MetaXpress Software (Molecular Devices). Count Nuclei or Cell Scoring application modules were used for nuclear count live/dead assessment, or evaluation of cell number positive for specific markers. Output measurements included spheroid width, spheroid area, average intensity for calcein AM or EthD-III, counts of all nuclei, and evaluation of average nuclear size and average intensities. In addition, calcein AM-positive cells were counted, and their area and intensity values were recorded. In addition to cell count, areas and intensities could be determined for live (EthD-III negative) and dead (EthD-III positive) cells. EC50 values were determined using a 4-parameter curve fit in SoftMax Pro 7 Software (Molecular Devices) (Sirenko, 2016). For the Cell Painting analysis, image analysis was done in the IN Carta image analysis software. To segment the organoids, a deep learning-based model was created based on the TL channel to segment individual organoids. Following segmentation, measurements that include intensity, shape, area, and texture were extracted from all 6 fluorescent channels. In total, 210 analytical features were obtained from each organoid.

### Example 8. Cell Painting Assay and Data Analysis

For the Cell Painting assay, the 3D TU-BcX-4IC organoids were labeled using a protocol modified from Bray et al. 2016, Nature protocols, 11(9), 1757-1774.

The plate was first incubated with MitoTracker DeepRed (500 nM) for 2 hours. The samples were fixed with 4% paraformaldehyde (PFA) in HBSS for 60 minutes. All wash steps were carried out by exchanging half the volume in each well with HBSS to minimize displacement of the organoids from the center of the well. Following fixation, samples were washed three times with HBSS. For permeabilization, samples were incubated with 0.1% Triton X-100 (in HBSS) for two hours at room temperature and washed with HBSS. The dyes were prepared in HBSS and 1% BSA (wt/vol) and incubated overnight with the following final concentrations: Hoechst (15 μg/ml), Concanavalin A-488 (250 μg/ml), Syto14 (7.5 μM), Phalloidin 750 (15 μl/ml), WGA (3.75 μg/ml).

Measurements from IN Carta were exported as a CSV file and uploaded into StratoMineR (Core Life Analytica), a web-based data analytics platform for hit picking and phenotypic clustering. Briefly, the dataset was normalized against the median of the samples to account for plate-plate variation. Data with a significant skewness (P<0.0001) was transformed to approximate a normal distribution. Feature scaling using robust Z-score was applied to the dataset.

Data reduction was achieved using principal component analysis (PCA). The results of the first 10 principal components were used in downstream analysis. For hit selection, the unsupervised option was selected with reference to the median of the negative controls (p<0.05). The Euclidean distance (Perlman et al., 2004) of all the vectors to the control was calculated. This approach reduces the data for each component to just one distance score which represents the phenotypic effect of the compound treatment relative to untreated samples. The median of the Euclidean distances for each well based on its replicates are calculated. Statistically significant hits were identified based on P<0.05. Hierarchical cluster analysis was performed on statistically significant hits based on the principal components using Ward's linkage criteria (k-means=8).

### Example 9. Lactate Secretion Assay

The metabolic response of tumoroids to treatment was determined by measuring lactate secretion in supernatants that were collected from treated and untreated 4IC tumoroids at various timepoints of drug treatment using the microfluidic device Pu MA System (Protein Fluidics, Inc., Burlingame, CA). Supernatant collection was done in the flowchip for each treatment condition in the following way: medium with drug was transferred from an adjacent reagent well of the flowchip to the sample well with tumoroid and incubated for 3 hours. After incubation, the media containing secreted lactate was transferred back to the reagent well it came from and replaced with fresh media with drugs from another well for the next 3-hour treatment. The cycle was repeated 5 times and resulted in collection of 5 supernatant samples. The first cycle was done with medium only (baseline secretion) followed by 4 treatment cycles for the total treatment duration of 12 hours. This approach allows dynamic monitoring of lactate secretion over the course of treatment.

The collected supernatants were stored in the flowchips until the end of the Pu MA protocol, then were collected and stored at −20° C. until further processing. The supernatant samples were analyzed for lactate levels using the luminescent Lactate-Glo assay (Promega). Lactate detection reagent was prepared according to the manufacturer's protocol. Supernatant samples were diluted 1:400 in PBS. 10 mL of the diluted supernatant was transferred to a solid white 384-well assay plate and 10 mL of lactate detection reagent was added to each well. Plates were incubated for 60 minutes at room temperature. Luminescence was measured using a GloMax plate reader (Promega). Each sample was measured in duplicate. Good luminescence signal levels and signal-to-noise ratios were achieved for this assay from single tumoroid samples. The statistical significance of comparison between groups was determined using one-way or two-way ANOVA. Post hoc tests were run to confirm where the differences occurred between groups. Differences with p<0.05 were considered statistically significant.

### Clauses

Clause 1. A method of phenotypic characterization of a three-dimensional (3D) target cell culture model, the method comprising
culturing the 3D target cell model in wells of a well plate for a first period of time;
staining the cultured 3D target cell model with three or more, four or more, five or more, or six or more dyes;
imaging the stained 3D target cell model; and
analyzing the images to quantify one or more phenotypic characteristics of the 3D target cell model.

Clause 2. The method of clause 1, wherein the imaging comprises
capturing using a digital imaging device, a first plurality of vertically spaced-apart images of the wells, each image having a different height along a Z axis, such that a volumetric image stack is generated with respect to the wells.

Clause 3. The method of clause 2, wherein respective Z-coordinates of sequential images in the volumetric image stack differ by at least approximately 1 micrometer.

Clause 4. The method of clause 3, wherein the respective Z-coordinates of sequential images in the volumetric image stack differ by less than approximately 50 micrometers, less than 40 micrometers, less than 30 micrometers, less than 25 micrometers, or less than 20 micrometers.

Clause 5. The method of clause 3 or 4, wherein the respective Z-coordinates of sequential images in the volumetric image stack differ by between 1 micrometer and 25 micrometers, 2 micrometers and 20 micrometers, or 3 micrometers and 15 micrometers.

Clause 6. The method of any one of clauses 1 to 5, wherein the three or more dyes comprise at least three dyes each specific for a different biomarker, cellular component or organelle selected from the group consisting of nuclear DNA, lysosomes, RNA, endoplasmic reticulum (ER), nuclei, nucleoli, cytoplasmic RNA, actin, Golgi apparatus, plasma membrane, mitochondria, and cytoskeleton.

Clause 7. The method of any one of clauses 1 to 6, wherein the three or more dyes are selected from the group consisting of a cell-permeant cell viability dye, a cell-impermeant dead cell nucleic acid stain, a bis-benzimide DNA stain, a E-cadherin stain, and a cell surface biomarker stain.

Clause 8. The method of any one of clauses 1 to 7, wherein the three or more dyes are selected from the group consisting of fluorescent dyes, luminescent dyes, and quantum dots, optionally wherein the three or more dyes comprise a dye-antibody conjugate, wherein the antibody is capable of specific binding to the selected biomarker, cellular component, or organelle.

Clause 9. The method of any one of clauses 1 to 8, wherein the imaging comprises illuminating the stained 3D target cell model with a multiplicity of lasers at different wavelengths suitable for excitation of the fluorescent dyes, optionally wherein images are acquired at different emission wavelengths for each of the fluorescent dyes, and optionally wherein each of the fluorescent dyes are specific for a different phenotypic characteristic.

Clause 10. The method of any one of clauses 1 to 9, wherein the staining comprises sequentially adding one or more of the three or more dyes to the 3D target cell model.

Clause 11. The method of any one of clauses 1 to 9, wherein the staining comprises simultaneously adding the three or more dyes to the 3D target cell model.

Clause 12. The method of any one of clauses 1 to 10, wherein the one or more phenotypic characteristics are selected from the group consisting of 3D target cell model size; diameter; area; disintegration; density; compactness; texture; integrity; optical density; shape; width; cell viability; ATP level; nuclei count; nuclear area; fluorescence intensity; total cell count; live cell count; dead cell count; cell area; projected cell area; number of viable cells; and cell number positive for a selected biomarker, cellular component, or organelle.

Clause 13. The method of clause 12, wherein the biomarker, cellular component, or organelle is selected from the group consisting of nuclear DNA, lysosomes, RNA, DNA, endoplasmic reticulum (ER), nuclei, nucleoli, cytoplasmic RNA, actin, Golgi apparatus, plasma membrane, mitochondria, and cytoskeleton.

Clause 14. The method of any one of clauses 1 to 13, further comprising monitoring one or more phenotypic characteristics of the 3D target cell model at one or more, two or more, three or more, or four or more time points during the culturing, optionally wherein the monitoring comprises imaging using transmitted light (TL).

Clause 15. The method of any one of clauses 1 to 14, further comprising fixing and optionally permeabilizing the cultured 3D target cell model.

Clause 16. The method of any one of clauses 1 to 15, wherein the method further comprises measuring one or more secreted factors in 3D target cell model supernatants.

Clause 17. The method of any one of clauses 1 to 16, further comprising treating the cultured 3D target cell model with one or more candidate compounds over a second period of time, optionally wherein the treating is before the staining.

Clause 18. The method of clause 17, wherein the treating comprises
exposing the cultured 3D target cell model to different doses of the one or more candidate compounds to obtain a dose-response curve for each of the one or more phenotypic characteristics,
optionally wherein the different doses include two or more, three or more, four or more, or five or more different doses.

Clause 19. The method of clause 18, wherein the analyzing comprises
calculating a phenotypic distance score for each candidate compound based on the dose-response curve for each of the one or more phenotypic characteristics, optionally wherein the phenotypic distance score is compared to an untreated control; and
identifying candidate compounds having a phenotypic distance score above a threshold value for each of the one or more phenotypic characteristics.

Clause 20. The method of any one of clauses 17 to 19, wherein the analyzing further comprises clustering the identified candidate compounds based on the phenotypic distance score for one or more phenotypic characteristics to create a phenotypic profile for the 3D target cell model.

Clause 21. The method of any one of clauses 1 to 20, wherein the 3D target cell model is selected from the group consisting of spheroid, tumoroid, organoid, and PDX-derived organoids.

Clause 22. The method of any one of clauses 1 to 21, wherein the 3D target cell model is derived from a patient tissue, tumor, biopsy sample, or a tumoroid fragment.

Clause 23. The method of clause 22, further comprising
obtaining isolated cells from a primary tumor of a patient;
cultivating the isolated cells to obtain two-dimensional (2D) cultivated cancer cells or passaging into murine models for expansion to provide xenograft cancer cells; and
forming the 3D target cell model from the 2D or xenograft cancer cells.

Clause 24. A method for selecting a drug therapy for therapeutic treatment of a subject in need thereof, the method comprising
culturing a 3D target cell model derived from the subject in wells of a well plate over a first period of time;
treating the cultured 3D target cell model with one or more candidate drugs over a second period of time;
staining the treated 3D target cell model with three or more dyes;
imaging the stained 3D target cell model; and
analyzing the images to quantify one or more phenotypic characteristics of the treated 3D target cell model.

Clause 25. The method of clause 24, further comprising
calculating a phenotypic distance score for each candidate drug for the one or more phenotypic characteristics compared to an untreated control; and
selecting one or more of the candidate drugs having a phenotypic distance score above a threshold value for therapeutic treatment of the subject.

Clause 26. The method of clause 24 or 25, wherein the imaging comprises
capturing using a digital imaging device, a first plurality of vertically spaced-apart images of the well, each image having a different height along a Z axis, such that a volumetric image stack is generated with respect to the well.

Clause 27. The method of clause 26, wherein respective Z-coordinates of sequential images in the volumetric image stack differ by at least approximately 1 micrometer.

Clause 28. The method of clause 27, wherein the respective Z-coordinates of sequential images in the volumetric image stack differ by less than approximately 50 micrometers, less than 40 micrometers, less than 30 micrometers, less than 25 micrometers, or less than 20 micrometers.

Clause 29. The method of clause 27 or 28, wherein the respective Z-coordinates of sequential images in the volumetric image stack differ by between 1 micrometer and 25 micrometers, 2 micrometers and 20 micrometers, or 3 micrometers and 15 micrometers.

Clause 30. The method of any one of clauses 24 to 29, wherein the treating comprises exposing the cultured 3D target cell model to different doses of the one or more candidate drugs to obtain a dose-response curve for each of the one or more phenotypic characteristics, optionally wherein the different doses include two or more, three or more, four or more, or five or more different doses.

Clause 31. The method of any one of clauses 24 to 30, wherein the analyzing comprises calculating the phenotypic distance score for each candidate drug based on the dose-response curve for each of the one or more phenotypic characteristics.

Clause 32. The method of any one of clauses 24 to 31, further comprising monitoring one or more phenotypic characteristics of the 3D target cell model before and at one or more, two or more, three or more, or four or more time points during the culturing and/or treating, optionally wherein the monitoring comprises imaging using transmitted light (TL).

Clause 33. The method of any one of clauses 24 to 32, further comprising fixing and optionally permeabilizing the treated 3D target cell model.

Clause 34. The method of any one of clauses 24 to 33, wherein the three or more dyes comprise at least three dyes each specific for a different biomarker, cellular component or organelle selected from the group consisting of nuclear DNA, lysosomes, RNA, endoplasmic reticulum (ER), nuclei, nucleoli, cytoplasmic RNA, actin, Golgi apparatus, plasma membrane, mitochondria, and cytoskeleton.

Clause 35. The method of clause 34, wherein the three or more dyes are selected from the group consisting of a cell-permeant cell viability dye, a cell-impermeant dead cell nucleic acid stain, a bis-benzimide DNA stain, a E-cadherin stain, and a CD cell surface biomarker stain.

Clause 36. The method of clause 34 or 35, wherein the three or more dyes are selected from the group consisting of fluorescent dyes, luminescent dyes, and quantum dots, optionally wherein the multiplicity of dyes comprise a dye-antibody conjugate, wherein the antibody is capable of specific binding to a selected biomarker, cellular component, or organelle.

Clause 37. The method of any one of clauses 24 to 36, wherein the imaging comprises illuminating the stained 3D target cell model with a multiplicity of lasers at different wavelengths suitable for excitation of the fluorescent dyes, optionally wherein images are acquired at different emission wavelengths for each of the fluorescent dyes, and optionally wherein each of the fluorescent dyes are specific for a different phenotypic characteristic.

Clause 38. The method of any one of clauses 24 to 37, wherein the staining comprises sequentially adding one or more of the three or more dyes to the treated 3D target cell model within the second period of time.

Clause 39. The method of any one of clauses 24 to 37, wherein the staining comprises simultaneously adding the three or more dyes to the treated 3D target cell model within the second period of time.

Clause 40. The method of any one of clauses 24 to 39, wherein the one or more phenotypic characteristics are selected from the group consisting of 3D target cell model size; diameter; area; disintegration; density; compactness; texture; integrity; optical density; shape; width; cell viability; ATP level; nuclei count; nuclear area; fluorescence intensity; total cell count; live cell count; dead cell count; cell area; projected cell area; number of viable cells; and cell number positive for a selected biomarker, cellular component, or organelle.

Clause 41. The method of clause 40, wherein the biomarker, cellular component, or organelle is selected from the group consisting of nuclear DNA, lysosomes, RNA, endoplasmic reticulum (ER), nuclei, nucleoli, cytoplasmic RNA, actin, Golgi apparatus, plasma membrane, mitochondria, plasma membrane, and cytoskeleton.

Clause 42. The method of any one of clauses 24 to 41, wherein the method further comprises measuring one or more secreted factors in 3D target cell model supernatants.

Clause 43. The method of any one of clauses 24 to 42, wherein the analyzing further comprises clustering the identified candidate compounds based on the phenotypic distance score for one or more phenotypic characteristics to create a phenotypic profile for the 3D target cell model.

Clause 44. The method of any one of clauses 24 to 43, wherein the 3D target cell model is selected from the group consisting of spheroid, tumoroid, organoid, and PDX-derived organoids.

Clause 45. The method of any one of clauses 24 to 44, wherein the 3D target cell model is derived from a patient tissue, tumor, biopsy sample, or a tumoroid fragment.

Clause 46. The method of any one of clauses 24 to 45, further comprising obtaining isolated cells from a primary tumor of a patient; cultivating the isolated cells to obtain two-dimensional (2D) cultivated cancer cells or passaging into murine models for expansion to provide xenograft cancer cells; and forming the 3D target cell model from the 2D or xenograft cancer cells.

We claim:

1. A method of phenotypic characterization of a three-dimensional (3D) target cell culture model, the method comprising
culturing the 3D target cell model in wells of a well plate for a first period of time;
staining the cultured 3D target cell model with three or more, four or more, five or more, or six or more dyes;
imaging the stained 3D target cell model; and
analyzing the images to quantify one or more phenotypic characteristics of the 3D target cell model.

2. The method of claim 1, wherein the imaging comprises
capturing using a digital imaging device, a first plurality of vertically spaced-apart images of the wells, each image having a different height along a Z axis, such that a volumetric image stack is generated with respect to the wells.

3. The method of claim 2, wherein respective Z-coordinates of sequential images in the volumetric image stack differ by at least approximately 1 micrometer.

4. The method of claim 3, wherein the respective Z-coordinates of sequential images in the volumetric image stack differ by less than approximately 50 micrometers, less than 40 micrometers, less than 30 micrometers, less than 25 micrometers, or less than 20 micrometers.

5. The method of claim 4, wherein the respective Z-coordinates of sequential images in the volumetric image stack differ by between 1 micrometer and 25 micrometers, 2 micrometers and 20 micrometers, or 3 micrometers and 15 micrometers.

6. The method of claim 1, wherein the three or more dyes comprise at least three dyes each specific for a different biomarker, cellular component or organelle selected from the group consisting of nuclear DNA, lysosomes, RNA, endoplasmic reticulum (ER), nuclei, nucleoli, cytoplasmic RNA, actin, Golgi apparatus, plasma membrane, mitochondria, and cytoskeleton.

7. The method of claim 6, wherein the three or more dyes are selected from the group consisting of a cell-permeant cell viability dye, a cell-impermeant dead cell nucleic acid stain, a bis-benzimide DNA stain, a E-cadherin stain, and a CD cell surface biomarker stain.

8. The method of claim 6, wherein the three or more dyes are selected from the group consisting of fluorescent dyes, luminescent dyes, and quantum dots, optionally wherein the three or more dyes comprise a dye-antibody conjugate, wherein the antibody is capable of specific binding to the selected biomarker, cellular component, or organelle.

9. The method of claim 8, wherein the imaging comprises
illuminating the stained 3D target cell model with a multiplicity of lasers at different wavelengths suitable for excitation of the fluorescent dyes, optionally
wherein images are acquired at different emission wavelengths for each of the fluorescent dyes, and optionally wherein each of the fluorescent dyes are specific for a different phenotypic characteristic.

10. The method of claim 1, wherein the staining comprises
sequentially adding one or more of the three or more dyes to the 3D target cell model.

11. The method of claim 1, wherein the staining comprises
simultaneously adding the three or more dyes to the 3D target cell model.

12. The method of claim 1, wherein the one or more phenotypic characteristics are selected from the group consisting of 3D target cell model size; diameter; area; disintegration; density; compactness; texture; integrity; optical density; shape; width; cell viability; ATP level; nuclei count; nuclear area; fluorescence intensity; total cell count; live cell count; dead cell count; cell area; projected cell area; number of viable cells; and cell number positive for a selected biomarker, cellular component, or organelle.

13. The method of claim 12, wherein the biomarker, cellular component, or organelle is selected from the group consisting of nuclear DNA, lysosomes, RNA, DNA, endoplasmic reticulum (ER), nuclei, nucleoli, cytoplasmic RNA, actin, Golgi apparatus, plasma membrane, mitochondria, and cytoskeleton.

14. The method of claim 1, further comprising
monitoring one or more phenotypic characteristics of the 3D target cell model at one or more, two or more, three or more, or four or more time points during the culturing, optionally wherein the monitoring comprises imaging using transmitted light (TL).

15. The method of claim 1, further comprising
fixing and optionally permeabilizing the cultured 3D target cell model.

16. The method of claim 1, wherein the method further comprises
measuring one or more secreted factors in 3D target cell model supernatants.

17. The method of claim 1, further comprising
treating the cultured 3D target cell model with one or more candidate compounds over a second period of time, optionally wherein the treating is prior to the staining.

18. The method of claim 17, wherein the treating comprises
exposing the cultured 3D target cell model to different doses of the one or more candidate compounds to obtain a dose-response curve for each of the one or more phenotypic characteristics,
optionally wherein the different doses include two or more, three or more, four or more, or five or more different doses.

19. The method of claim 18, wherein the analyzing comprises
calculating a phenotypic distance score for each candidate compound based on the dose-response curve for each of the one or more phenotypic characteristics, optionally wherein the phenotypic distance score is compared to an untreated control; and
identifying candidate compounds having a phenotypic distance score above a threshold value for each of the one or more phenotypic characteristics.

20. The method of claim 19, wherein the analyzing further comprises
clustering the identified candidate compounds based on the phenotypic distance score for one or more phenotypic characteristics to create a phenotypic profile for the 3D target cell model.

21. The method of claim 1, wherein the 3D target cell model is selected from the group consisting of spheroid, tumoroid, organoid, and PDX-derived organoids.

22. The method of claim 21, wherein the 3D target cell model is derived from a patient tissue, tumor, biopsy sample, or a tumoroid fragment.

23. The method of claim 22, further comprising
obtaining isolated cells from a primary tumor of a patient;
cultivating the isolated cells to obtain two-dimensional (2D) cultivated cancer cells or passaging into murine models for expansion to provide xenograft cancer cells; and
forming the 3D target cell model from the 2D or xenograft cancer cells.

24. A method for selecting a drug therapy for therapeutic treatment of a subject in need thereof, the method comprising
culturing a 3D target cell model derived from the subject in wells of a well plate over a first period of time;
treating the cultured 3D target cell model with one or more candidate drugs over a second period of time;
staining the treated 3D target cell model with three or more dyes;
imaging the stained 3D target cell model; and
analyzing the images to quantify one or more phenotypic characteristics of the treated 3D target cell model.

25. The method of claim 24, further comprising
calculating a phenotypic distance score for each candidate drug for the one or more phenotypic characteristics compared to an untreated control; and
selecting one or more of the candidate drugs having a phenotypic distance score above a threshold value for therapeutic treatment of the subject.
